(19) United States
(12) Patent Application Publication
(10) Pub. No.: US 2023/0071306 A1
(43) Pub. Date: Mar. 9, 2023

(54) SYSTEMS AND METHODS FOR DELIVERING TARGETED THERAPY

(71) Applicant: Intuitive Surgical Operations, Inc., Sunnyvale, CA (US)

(72) Inventors: KYLE R. MILLER, San Jose, CA (US); Joseph D. Bogusky, San Jose, CA (US); Duane W. Marion, Scottsdale, AZ (US); Ian E. McDowall, Woodside, CA (US); Tabish Mustufa, Sunnyvale, CA (US); Dinesh Rabindran, San Jose, CA (US); Benjamin J. Schoettgen, Los Gatos, CA (US); Jignesh Shah, Sunnyvale, CA (US); Tao Zhao, Sunnyvale, CA (US)

(21) Appl. No.: 17/800,279

(22) PCT Filed: Feb. 18, 2021
(86) PCT No.: PCT/US2021/018493

§ 371 Date: Aug. 17, 2022

Related U.S. Application Data
(60) Provisional application No. 62/979,948, filed on Feb. 21, 2020.

(51) Int. Cl. A61B 90/00; A61B 18/14

(52) U.S. Cl. CPC A61B 90/39; A61B 18/1492; A61B 2018/00577

(57) ABSTRACT

A computer-assisted medical device is configured and used to endoluminally navigate to a location in the gastrointestinal system and there treat certain body lumen wall areas while avoiding other body lumen wall areas. Embodiments ablate the inner mucosal layer and sub-mucosal nerve plexus of the stomach, duodenum and jejunum to effect treatment of insulin resistance and metabolic disorders, such as Type II diabetes (T2D), polycystic ovarian syndrome (PCOS), non-alcoholic steatohepatitis (NASH), non-alcoholic fatty liver disease (NAFLD), congestive heart failure (CHF) and obstructive sleep apnea (OSA). Various sensors are used to assist a clinical operator to navigate from the mouth through the pyloric sphincter and into and through the duodenum and/or jejunum. Various sensors are used to map and identify portions of the duodenum and/or jejunum. Various lumen wall ablation devices and methods are described. Various post-treatment assessments are described.

### CROSS-REFERENCE TO RELATED APPLICATIONS

This application claims the benefit of U.S. Provisional Application 62/979,948 filed Feb. 21, 2020, which is incorporated by reference herein in its entirety.

### FIELD

Aspects of the present disclosure are directed to systems and methods for care during medical or surgical procedures, and in particular to accessing and providing therapy to intra-body target tissue during a medical or surgical procedure.

### BACKGROUND

Minimally invasive medical techniques are intended to reduce the amount of tissue damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator inserts minimally invasive medical instruments (diagnostic, therapeutic) to reach a target tissue location. It is desirable to provide a delivery system that will help guide a user to safely and accurately place medical and surgical devices near target tissue during a medical procedure. It is also desirable to deliver agents and molecules prior to the procedure via orally ingested or intravenously administered routes in order to aid with anatomy identification and mapping.

### SUMMARY

Various embodiments of the present disclosure are summarized by the claims that follow the description. Other embodiments may be claimed at a later time. Consistent with some embodiments, a method for accessing and treating a target tissue within a patient anatomy is performed by a processor. The method comprises creating a first model of the patient anatomy by using information from one or more localization sensors and one or more imaging sensors or agents delivered pre-procedure to aid with registration and mapping. The one or more localization sensors and the one or more imaging sensors are coupled to a medical device being delivered through the patient anatomy to the target tissue. The method also comprises receiving data identifying one or more anatomical area of interest within the patient anatomy and creating a second model based on the first model and the one or more identified anatomical area of interest. The method also comprises displaying the second model and performing a medical procedure on the one or more anatomical areas of interest.

Consistent with some embodiments, a medical system for accessing a patient anatomy comprises a medical device including a main lumen and a localization sensor providing position of the medical device from both external and internal adjuncts and markers. The medical system also comprises an imaging sensor configurable to provide an image from a distal portion of the medical device and a processor in communication with the localization sensor and the imaging sensor. The processor is configured to generate a first model using information from the localization sensor and the imaging sensor, to receive data for identifying anatomic features of interest within the patient anatomy, and to update the first model to generate a second model that includes the anatomic features of interest. The medical system also includes a display for displaying at least one of the first model and the second model.

Consistent with some embodiments, the systems and methods described in the present disclosure include (i) navigation and control of medical devices to one or more target areas; (ii) visualization and mapping of the one or more target areas and real-time tissue-characterization in the target areas; (iii) selective treatment within target areas while protecting sensitive areas; and (iv) tracking and visualization of the applied treatment and area of anatomy treated, and optionally measurement and/or characterization of treatment effectiveness, including treatment amount and area. By way of example, the system and methods can be applied to a patient's digestive system, such as treatment in the stomach, duodenum, and/or jejunum for the treatment of diabetes and other metabolic diseases.

It is to be understood that both the foregoing general description and the following detailed description are exemplary and explanatory in nature and are intended to provide an understanding of the present disclosure without limiting the scope of the present disclosure. And so, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a cross-sectional view of a medical instrument system according to some embodiments.

FIG. 2B is a simplified diagram of a medical instrument with an extended medical tool according to some embodiments.

FIG. 3 is a flowchart of an example medical procedure according to some embodiments.

FIG. 4 illustrates a medical system inserted into a patient anatomy according to some embodiments.

FIG. 5 is a detailed view of a patient anatomy according to some embodiments.

FIG. 6 illustrates a medical system inserted into a patient anatomy according to some embodiments.

FIGS. 7A-7C illustrate a medical system inserted into a patient anatomy according to some embodiments.

FIG. 8 is a simplified diagram of a medical system according to some embodiments.

FIG. 9 is a detailed view of a patient anatomy according to some embodiments.

FIG. 10 illustrates a medical system inserted into a patient anatomy according to some embodiments.

FIG. 11A illustrate a medical system inserted into a patient anatomy according to some embodiments.

FIG. 11B illustrate a medical system inserted into a patient anatomy according to some embodiments.

FIG. 12A illustrates a medical system inserted into a patient anatomy according to some embodiments.

FIG. 12B illustrates a medical system inserted into a patient anatomy according to some embodiments.

FIG. 13A-13C are a simplified diagrams of medical instruments according to some embodiments.

FIG. 14 illustrates a medical system inserted into a patient anatomy according to some embodiments.

FIGS. 15A-15B illustrate medical systems inserted into a patient anatomy according to some embodiments.

FIG. 16 illustrates a medical system inserted into a patient anatomy according to some embodiments.

FIG. 17 illustrates a medical system inserted into a patient anatomy according to some embodiments.

FIG. 18 illustrates a medical system inserted into a patient anatomy according to some embodiments.

FIG. 19 illustrates a medical system inserted into a patient anatomy according to some embodiments.

FIG. 20 illustrates a medical system inserted into a patient anatomy according to some embodiments.

FIG. 21 illustrates a medical system inserted into a patient anatomy according to some embodiments.

FIG. 22 illustrates a medical system inserted into a patient anatomy according to some embodiments.

FIG. 23 illustrates a medical system inserted into a patient anatomy according to some embodiments.

FIG. 24 illustrates a medical system inserted into a patient anatomy according to some embodiments.

FIGS. 25A-25C illustrate simplified diagrams of sensor feedback according to some embodiments.

Embodiments of the present disclosure and their advantages are best understood by referring to the detailed description that follows. It should be appreciated that like reference numerals are used to identify like elements illustrated in one or more of the figures, which illustrate embodiments of the present disclosure without limitation.

### DETAILED DESCRIPTION

In the following description, specific details are set forth describing some embodiments consistent with the present disclosure. Numerous specific details are set forth in order to provide a thorough understanding of the embodiments. It will be apparent to one skilled in the art, however, that some embodiments may be practiced without some or all of these specific details. The specific embodiments disclosed are meant to be illustrative but not limiting. One skilled in the art should realize other elements exist that, although not specifically described, are within the scope and the spirit of this disclosure. In addition, to avoid unnecessary repetition, one or more features shown and described in association with one embodiment may optionally be incorporated into other embodiments unless specifically described otherwise or if the one or more features would make an embodiment non-functional.

In some instances, well-known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three translational mechanical degrees of freedom (DOFs) along Cartesian X-, Y-, and Z-coordinates). As used herein, the term “orientation” refers to the rotational placement of an object or a portion of an object in a three-dimensional space (e.g., three rotational mechanical DOFs around Cartesian X-, Y-, and Z-coordinates; sometimes referred to as pitch, roll, and yaw as defined in relation to the axes). As used herein, the term “pose” refers to the position of an object or a portion of an object in at least one translational DOF and to the orientation of that object or portion of the object in at least one rotational DOF (up to six total DOFs). As used herein, the term “shape” refers to a set of poses, positions, or orientations measured along an object.

Minimally invasive procedures typically include introducing a minimally invasive medical or surgical instrument through a natural orifice in a patient anatomy or through one or more surgical incisions and navigating the instrument through patient anatomy to access anatomical target tissue for diagnosis or treatment. In one embodiment, a medical instrument is used to access various areas of patient anatomy, such as within a patient digestive system, respiratory system, renal system, circulatory system, and/or reproductive system. In some cases, an instrument with a working lumen is navigated through anatomy, positioned at the anatomical target, and then used as a delivery conduit for one or more additional diagnostic and/or therapeutic medical devices. Examples of medical devices include an endoscope, an ablation device, a biopsy tool, a surgical tool, an ultrasound probe, and/or the like. The minimally invasive medical instruments are optionally flexible, semi-rigid or rigid. Navigation of the instruments can be manual, automatic under computer control, or a combination of manual with computer assistance. Navigation optionally occurs under endoscopic guidance, under external imaging guidance such as fluoroscopy, or a combination of both.

In some cases, depending on the type of medical procedure to be performed and/or the anatomy to be navigated, it can be difficult to identify target tissue or sensitive anatomy that should be avoided. Pre-operative images (CT, MRI, Ultrasound. Upper GI small bowel follow though based on a series of X-rays and the like) can be used to create a model of the anatomy, but in many cases imaging technology may not be available, capturing pre-operative imaging data may not be a standard practice for a planned procedure, models may not adequately display the target tissue or sensitive anatomy, or anatomy may shift or changes in the anatomy may occur between the time of imaging and the time of the procedure due to positioning and/or the body's response to nerve signals (parasympathetic and sympathetic). Thus it is helpful to generate accurate models (which may function as maps) of anatomy, plan medical procedures according to the models, and display the models with planned procedural information for guidance as an aid to diagnose, visualize, and/or treat different disease states or conditions. It may also be helpful, especially in cases requiring treatment of tissue, to map overlap and/or proximity of successive treatment areas to allow for successive treatments along an anatomical lumen and have desired overlap of treatment areas or avoid overlap of treatment areas. For example, if radiofrequency (RF) ablation is used, no overlap is desired because it may harm or result in narrowing of the anatomical lumen and underlying structures within the wall of the intestines, such as causing stenosis in the post procedure course of the patient. In other treatments, a fixed overlap area optionally can be defined if desired. And, in still other situations a combination of one or more defined overlap and non-overlap regions may be defined. Thus, with the use of position sensors in conjunction with visual confirmation, models of anatomy can be generated, medical procedures can be planned, and models can be updated based on nerve signaling, perfusion of the hollow viscous structure, patient positioning, disease state and target anatomy.

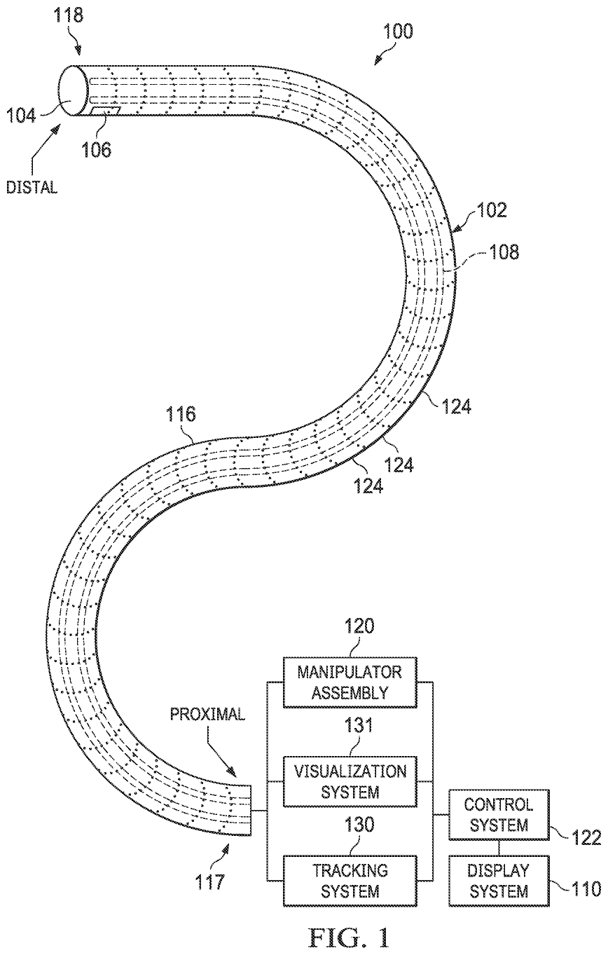

FIG. 1 is a medical system 100 in accordance with the present disclosure. Medical instrument system 100 includes an elongate device 102 having a flexible body 116 and a main lumen 104 (also referred to as a working lumen, main channel, or working channel) and a localization sensor or set of localization sensors, which optionally may be integrated into a wall of the elongate device 102. Alternatively, the localization sensor or sensors may be slideably disposed in main lumen 104 or in another lumen (not shown), or they may be otherwise integrated into the body of elongate device 102. The flexible body 116 extends between a distal end 118 and a proximal end 117. The elongate device 102 optionally may be coupled to or in communication with a variety of systems, including a kinematic arm manipulator assembly 120, a visualization system 131, and a tracking system 130. Either directly or via at least one of the manipulator assembly 120, the visualization system 131, and/or the tracking system 130, the elongate device 102 may be coupled to or in communication with a control system 122 and a display system 110.

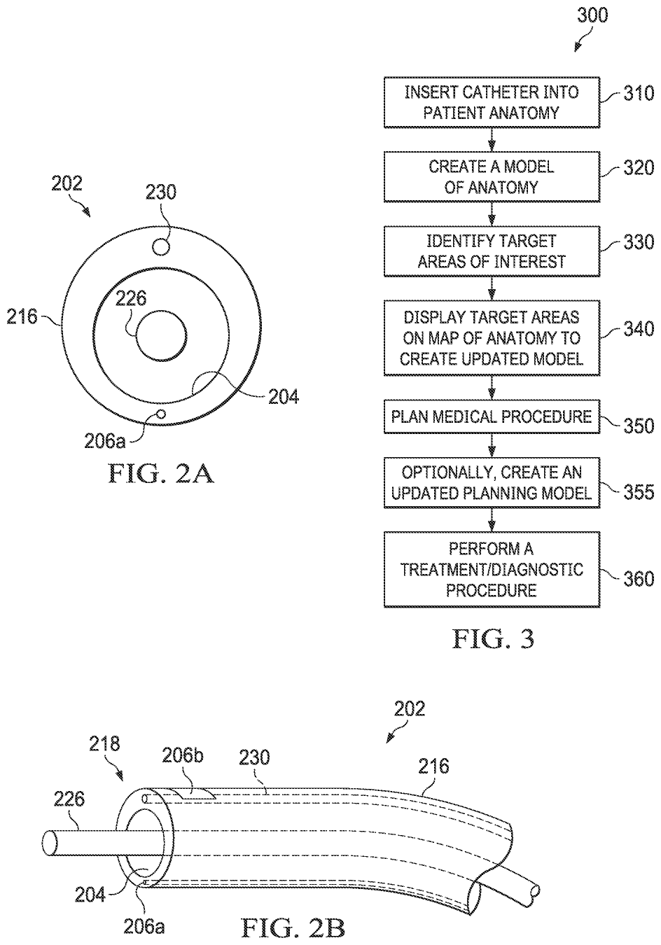

The main lumen 104 may be used as an open delivery channel for delivery of one or more various devices (described below with reference to FIGS. 2A and 2B), as well as devices used to aid in steering of the elongate device 102, such as guide wires, overtubes, and/or the like. The localization sensor(s) optionally may also be integrated within a second device inserted into the main lumen 104 of the elongate device 102 and used as part of the system for localization and navigation. The second device may be removed during treatment, especially if a treatment device includes separate localization sensor(s). Localization sensors can include sensors (e.g., a single position sensor 106, a plurality of position sensors distributed along the length of the elongate device 102, a shape sensor 108, and/or an imaging sensor) coupled to the tracking system 130 for receiving and processing sensor data and information for determining the position, orientation, speed, velocity, pose, and/or shape of distal end 118 and/or of one or more lengths along the flexible body 116. Tracking system 130 may optionally be implemented as hardware, firmware, software, or a combination thereof, any of which interact with or are otherwise executed by one or more computer processors.

Tracking system 130 may optionally track one or more positions and/or orientations of distal end 118 and/or one or more of segments 124 of the flexible body 116 by using a shape sensor 108. Shape sensor 108 may optionally include an optical fiber aligned with flexible body 116 (e.g., provided within an interior channel (not shown) or mounted externally). The optical fiber of shape sensor 108 forms a fiber optic bend sensor for determining the shape of flexible body 116. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005)(disclosing “Fiber optic position and shape sensing device and method relating thereto”); U.S. patent application Ser. No. 12/047,056 (filed on Jul. 16, 2004)(disclosing “Fiber-optic shape and relative position sensing”); and U.S. Pat. No. 6,389,187 (filed on Jun. 17, 1998)(disclosing “Optical Fibre Bend Sensor”), which are all incorporated by reference herein in their entireties. Sensors in some embodiments may employ other suitable strain sensing techniques, such as Rayleigh scattering, Raman scattering. Brillouin scattering, and Fluorescence scattering. In some embodiments, the shape of the elongate device may be determined by using other techniques. For example, a history of the distal end pose of flexible body 116 can be used to reconstruct the shape of flexible body 116 over the interval of time. In some embodiments, tracking system 130 may optionally and/or additionally track distal end 118 by using a position sensor system 106. Position sensor system 106 may be a component of an electromagnetic (EM) sensor system, with position sensor system 106 including one or more electrically conductive coils subjected to an externally generated electromagnetic field. Each coil of the EM sensor system then produces an induced electrical signal having characteristics that depend on the position and orientation of the coil relative to the externally generated electromagnetic field. In some embodiments, position sensor system 106 optionally may be configured and positioned to measure DOFs in various coordinate systems. For example, three position coordinates X, Y, Z and three orientation angles indicating pitch, yaw, and roll at a base point, or five degrees of freedom, or three position coordinates X, Y, Z and two orientation angles indicating pitch and yaw at a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

In some embodiments, tracking system 130 may alternately and/or additionally rely on historical pose, position, or orientation data stored for a known point of an instrument system along a cycle of alternating motion, such as breathing generated by the phrenic nerve and diaphragm contraction or intestinal peristaltic waves generated by the enteric nervous system. This stored data may be used to develop shape information about flexible body 116. In some examples, a series of positional sensors (not shown), such as EM sensors similar to the sensors in position sensor 106 may be positioned along flexible body 116 and then used for shape sensing. In some examples, a history of data from one or more of these sensors taken during a procedure may be used to represent the shape of elongate device 102, particularly if an anatomic passageway is generally static.

FIGS. 2A and 2B illustrate a simplified diagram of an end view and a side view, respectively, of another example of an elongate device 202 including flexible body 216 having distal end 218, main lumen 204, and localization or position sensor(s) 206a and 206b. Elongate device 202 is generally equivalent in construction and function as elongate device 102 except where described herein. In some embodiments, position sensor 206a may include an optical fiber shape sensor integrated along the length of elongate device 202. In some embodiments, position sensor 206b includes a single position sensor, such as an EM sensor, optical encoder, and/or the like, as shown. In some embodiments, position sensor 206a is positioned in a different circumferential position as 206b as illustrated in FIGS. 2A and 2B. In some embodiments, position sensor 206a is in a substantially similar circumferential position as position sensor 206b. In some embodiments, elongate device 202 includes a plurality of position sensors 206b positioned along the length of, and/or at different locations around the circumference of, the elongate device 202. In some embodiments, elongate device includes a plurality of position sensors 206a and a plurality of position sensors 206b. In some embodiments, elongate device 202 includes only a single or plurality of position sensors 206a or elongate device 202 includes a single or plurality of position sensor 206b.

In another example, position sensor 206a is included along the length of a medical instrument 226, such that position, orientation, pose, and/or shape of the medical instrument 226 can be measured and then confirmed using imaging sensor 230 embedded within a wall or end of elongate device 202. Elongate device 202 may include imaging sensor 230, such tool based imaging devices for providing intraoperative images, for example endoscopic cameras (singular or plurality of cameras), endoluminal or intravascular ultrasound, optical coherence tomography (OCT) device, confocal microscopy and/or the like. Elongate device 202's position and/or orientation can be determined based on information from the imaging sensor 230 and/or from position sensor(s) 206a, 206b.

The medical instrument 226 may be slidably disposed within the main lumen 204 of the elongate device 202 and extended from the distal end 218. In some embodiments, medical instrument 226 is used for diagnostic or therapeutic procedures such as surgery, biopsy, ablation, imaging, illumination, irrigation, retraction, dissection, excision, exposure or suction. Medical instrument 226 can be deployed through main lumen 204 of flexible body 216 and used at a target location within the anatomy. Medical instrument 226 may include, for example, image capture probes, biopsy instruments, ablation elements (e.g. including ablation probes, laser ablation fibers, or ablation balloons), and/or other diagnostic or therapeutic tools. Medical tools may include end effectors having a single working member, such as a scalpel, a blunt blade, an optical fiber, an electrode, a hydrojet and/or the like. Other end effectors may include one or more movable components, for example, forceps, graspers, retractors, dissectors, scissors, clip appliers, advanced energy, staplers, micro-staplers, snares and/or the like. Still other end effectors may further include electrically activated end effectors, such as electrosurgical electrodes, transducers, sensors, and/or the like. In various embodiments, medical instrument 226 is a biopsy or tissue probe instrument, which may be used to remove sample tissue, characterize tissue in real-time or a sampling of cells from a target anatomic location. In various embodiments, medical instrument 226 is an instrument used to affect tissue on a body lumen wall, such as via various forms of ablation or tissue alteration.

Medical instrument 226 may be visualized by using imaging sensor 230 (e.g., an image capture probe) that includes a distal portion with a stereoscopic or monoscopic camera at or near distal end 218 of flexible body 216. The camera is used for capturing image data (including video image data) that are then processed by an imaging system, such as visualization system 131, for display and/or provided to tracking system, such as tracking system 130. The processed images are output to a clinical operator or are used by the system's control system to support tracking of distal end 218 and/or a portion of flexible body 216 along the length of flexible body. The imaging sensor may include a cable coupled to the camera for transmitting the captured image data. In some examples, the imaging sensor 230 may be a fiber-optic bundle, such as a fiberscope, that couples to visualization system 131. The imaging sensor may be single or multi-spectral, for example capturing image data in one or more of the visible, infrared, and/or ultraviolet spectrums. The imaging sensor 230 can be any type of minimally invasive tool providing intraoperative images, for example an endoscopic probe, an OCT probe, or an ultrasound probe (endoluminal or intravascular). Or, the imaging sensor 230 may include a combination of any number of endoscopic, OCT, antennas, electrodes, other electromagnetic measurement devices, and/or ultrasound probes. Target tissue and various anatomical structures can be identified visually in endoscopic camera images as well as in, e.g., ultrasound, confocal microscopy or OCT images. Tissue may be identified as ablated or non-ablated tissue in various ways, such as by measuring impedance of the tissue, temperature changes and monitoring a change in impedance or comparing impedance measurements against pre-determined impedance thresholds. Tissue may alternatively be identified as ablated or non-ablated by imaging either with a camera like device (e.g., using endoscopic camera images) or a scanning type technology such as OCT, confocal microscope, infrared temperature mapping, thermocoupler or ultrasound, or a combination thereof.

In some embodiments, the imaging sensor 230 may be slideably disposed through main lumen 204, slideably disposed within a secondary lumen offset from main lumen 204, fixed within the secondary lumen, integrated into a wall of the elongate device 202, or disposed external to elongate device 202. Alternatively, medical instrument 226 may itself be the image capture probe. Medical instrument 226 may be advanced from the distal opening of main lumen 204 to perform the procedure and then retracted back into the channel when the procedure is complete. Medical instrument 226 may be removed from the proximal end of flexible body 216 or from another optional instrument port (not shown) located along flexible body 216. In some embodiments flexible body 216 is not flexible but rigid or semi-rigid, and it optionally transitions between one or more of the rigid, semi-rigid, and flexible states.

In some embodiments, a separate sheath, steerable or non-steerable, is deployed over elongate device 202. In some embodiments, the sheath is a passive sheath with an integrated optical camera or vision probe. This vision-enabled sheath provides additional functionality for the device and leaves main lumen 204 available for other uses. In some embodiments, the passive sheath couples to the elongate body by using a locking mechanism or keying mechanism, e.g., a proximal locking mechanism, a distal locking mechanism, that keys the passive sheath and elongate body along the length or at a distal section of sheath and elongate body. In alternative embodiments, the passive sheath couples to the elongate body by using a keyed interface along the length of, or at a distal end portion of, the passive sheath or the elongate body.

In some examples, as described in detail below, the imaging sensor alone or in combination with other components of the medical instrument system 100 includes one or more mechanisms for cleaning one or more lenses of the imaging sensor when the one or more lenses become partially and/or fully obscured by fluids, bubbles, inorganic material, organic material, intestinal secretions, enzymatic secretions, biliary acids and/or other materials encountered by the distal end of the imaging sensor. In some examples, the one or more cleaning mechanisms may optionally include an air and/or other gas delivery system that is usable to emit a puff of air and/or other gasses to blow the one or more lenses clean. Examples of the one or more cleaning mechanisms are discussed in more detail in International Publication No. WO/2016/025465 (filed Aug. 11, 2016)(disclosing “Systems and Methods for Cleaning an Endoscopic Instrument”); U.S. patent application Ser. No. 15/508,923 (filed Mar. 5, 2017)(disclosing “Devices, Systems, and Methods Using Mating Catheter Tips and Tools”); and U.S. patent application Ser. No. 15/503,589 filed Feb. 13, 2017) (disclosing “Systems and Methods for Cleaning an Endoscopic Instrument)”, each of which is incorporated by reference herein in its entirety. The imaging system may be implemented as hardware, firmware, software, or a combination thereof, which interact with or are otherwise executed by one or more computer processors, which may include the processors of a control system.

In some examples, the elongate device 202 is used to accurately navigate to target tissue. Once present at the target tissue, the elongate device 202 may be held in a stationary position and orientation and the medical instrument 226 may be delivered through the main lumen 204 of the elongate device 202 or the medical instrument 226 may be coupled to the elongate device 202 and controllably articulated for visualization, diagnostics, and/or treatment. The medical instrument 226 may include an imaging device (endoscopic camera, ultrasound transducer, etc.), a diagnostic device, and/or a treatment device.

FIG. 3 is a flowchart of an example medical procedure 300 in accordance with the present disclosure. Medical procedure 300 generates a model of patient anatomy, and the model may be used to plan areas of treatment, and/or used for treatment of the target tissue. At process 310, an elongate device, such as elongate device 102, is introduced into anatomy to be navigated towards an anatomical target. The elongate device 102 can be used to access various areas of patient anatomy, such as within a patient digestive system, respiratory system, renal system, and/or reproductive system, to visualize, diagnose, and/or treat different disease states or conditions. In one embodiment, elongate device 102 is inserted into a natural orifice in a patient anatomy or through one or more surgical incisions and navigated to the anatomical target. In one example, the surgical incision is made to provide direct access to the target anatomy through a more direct path.

At process 320, an anatomic model or map of patient anatomy can be created. In one embodiment, the model is generated from data collected as elongate device 102 is navigated through an anatomical path towards the anatomical target. The data can be provided by one or more of position, localization, and imaging sensors, such as shape sensor 108, position sensor 106, and imaging sensor 230 coupled to elongate device 102. This real time sensing provides for real time localization, orientation, and/or position of a distal portion of the flexible body as well as the shape of the flexible body and images of internal surfaces of lumens, vessels, and/or organs. In some embodiments, a processor coupled to or provided with (racking system 130 may collect position and/or localization data representing the distal end 118 of the elongate device 102 as it is driven through anatomy, thereby providing a centerline of the anatomy as device 102 advances. The model may then be built including diameters of the of the anatomy by using either standard anatomical human data, using elongate device 102 and localization sensing to touch anatomical walls to determine area or volume, and/or data from imaging sensor 230 where diameter can be determined using image-based methods.

In some embodiments, elongate device 102 is routed within an internal region, and the distal end 118 of the elongate device 102 is positioned past the target tissue. Once past the target tissue, a shape sensor, such as shape sensor 108 or a plurality of position sensors 106 positioned down a length of the flexible body 116, can be used to measure a partial or full shape of a length of the elongate device 102. The model of the area surrounding the target tissue may then be rendered based on the partial or full shape of the elongate device 102, data gathered during navigation of elongate device 102, imaging data collected during navigation of elongate device 102 (e.g., endoscopic or ultrasound data), or any combination of all data.

In some embodiments, the model is initially generated using a generic anatomic model of average human anatomy, possibly chosen based on some heuristics, such as patient weight, height, sex and age. In another embodiment, the initial model is generated from pre-operative scans from imaging data obtained from a CT, PET CT, MRI, DICOM, ultrasound, x-ray, fluoroscopic images or prior small bowel enteroscopy data. In some cases, the pre-operative scans are taken weeks prior to a procedure, and so anatomy can shift, and imaging data may not accurately display all anatomical areas of interest, depending on the quality of the imaging equipment and the type of tissue or anatomical structure of interest. Thus, the initial model may then be altered, supplemented, or merged with data collected in real time while the elongate body is navigated through anatomy, e.g., position/location data gathered during navigation, endoscopic camera data correlated to positional data, and/or intravascular or endoluminal ultrasound data, to more accurately display the patient anatomy as previously described above.

At process 330, one or more specific anatomical target areas of interest are identified within the model generated in process 320. The areas may include anatomical structures within the body to be used as landmarks during navigation of the device; areas identified for treatment, such as lesions, tumors, or diseased tissue; sensitive structures or anatomy to be avoided during treatment, which may be located in close proximity to the treatment areas; and/or artificially identified locations, such as a previously placed real or synthetically generated markers within the tissue.

In some embodiments, the identification of landmarks can be user-defined, such as by visually identifying the landmark using a live direct endoscopic view or under live fluoroscopic guidance, and having the operator press a button when the elongate device is adjacent to or touching a landmark. The user can provide input to identify the type of landmark, by the control system. Software can compensate for a distance from the distal tip of the catheter/camera/endoscope to the landmark if the focal length of the camera and the visual appearance of the landmark are known. In some embodiments, identification of the landmark can be vision-based and automated, in which the image (e.g., the endoscopic image) is identified automatically, such as by comparing the image to a library of images of anatomical features or is identified by the approximate location and shape of the anatomical feature.

In some embodiments, ultrasound is used to determine depth tissue of interest, such as lesions, tumors, and/or diseased tissue such as hyperplastic tissue. An ultrasound transducer integrated into the elongate device or treatment device, or integrated into a probe delivered through the elongate device, may be used to capture ultrasound data. With localization sensors provided within the elongate device, the location of the ultrasound image can be correlated to the location of the elongate device and an exact location, as well as the depth, of the tissue of interest can be determined. Further, pulsed ultrasonic waves can be transmitted non-invasively to the tissue via an internal or external transducer to create cavitation and tissue ablation for targets of interest.

At process 340, the target area(s) of interest identified in process 330 are then displayed or rendered on the model of the anatomy generated from process 320 so as to create an updated model. In some embodiments, if ultrasound is used to identify depth of target tissue, the target tissue can be displayed at a measured location within the model and at a detected depth based on ultrasound data. In some embodiments, the model is a 3D model, and so the location and depth of the tissue target can be accurately displayed on the model and highlighted with a specific color, shade, hue, and/or transparency to visibly distinguish target and healthy tissue.

At process 350, the updated model is used to plan a medical procedure that includes determining a navigational path to an anatomic or ablation target and/or creating a treatment plan. The medical procedure plan can then be saved within a control system and/or used to provide additional detail or further update the updated model generated during process 340.

In one embodiment, the treatment plan includes the type of treatment including biopsy, diagnosis, dissection, delivery of chemicals, ablation, ultrasonic waves and/or the like. Treatment plans optionally include determining treatment parameters, such as treatment location, size of treatment area, treatment depth, number of treatments, and spacing of treatments. In some embodiments, treatment zones are planned so that they are spaced in a particular fashion or pattern, such as helically, spiraled or circumferentially. In one example, a treatment plan is created to ablate an anatomical target, such as a tumor, a nodule, or a lumen wall area. Accordingly, the treatment plan can specify location of ablative energy delivery, size and depth of ablation zones, number of ablation zones, and/or an ablation pattern.

In some embodiments, treatment power settings can be established at varied levels based on distance to sensitive anatomy, type of tissue being treated, and/or status of treatment. A treatment power setting optionally can be chosen based on depth of the diseased tissue. Power can be varied among treatment areas if ultrasound has identified deeper diseased tissue in some areas. To tailor treatment to one or more particular areas of interest, power can be scaled within a range from a maximum power to zero, or if power is set to a particular level then a threshold treatment distance can be established for safety. In some embodiments, power can be reduced farther away from the central area of target tissue to be treated so as to treat (e.g., ablate) a margin around target tissue. In some embodiments, circumferential ablations may be of alternating depths, e.g., power is modulated, increased and decreased for adjacent ablations to minimize the chance of stenosis or other damage to vessels. In some embodiments, power can be decreased as ablation continues. e.g., as tissue becomes more desiccated, tissue is burned, and/or tissue cells are killed. In some embodiments, other types of variable power ablation patterns may be created to most effectively treat tissue via active feedback relay and display.

The plan for the medical procedure can include locating sensitive areas and displaying locations which may require protection during a treatment procedure. For example, locations for deployment of a protective device can be determined and saved as part of the medical procedure plan. Similarly, locations where substances, such as gas, hetastarch, saline or water, can be applied to cool and protect the sensitive areas can be identified.

In some embodiments, while the model is being generated from data collected as the device is routed through anatomy as previously described, the device centerline path, or the body lumen centerline, or both, can be saved and displayed in the model as a navigation path to the anatomical target. The navigation path can be used if multiple treatment passes are to be used, or if the device is removed from patient anatomy and the operator needs to access the anatomical target in a follow up procedure or by using a different medical device. The anatomical model is optionally updated as described above during subsequent navigations.

In some embodiments, the treatment plan and/or updated treatment plan can be automatically created based on the model or updated model, and empirical data of ablation size, depth, and power requirements are correlated with tissue type and type of anatomical target (e.g., tumor or other hyperplastic tissue). In other embodiments, the treatment plan can be manually created by the operator using the model for guidance. In some embodiments, power settings and locations for ablations may be determined manually by looking at the model and determining if lesser or greater ablation is needed in certain areas or a certain ablation pattern is needed. In some embodiments, a combination of software-defined and manual determinations are made, e.g., the software sets an ablation pattern with power settings and the user alters the software's proposed plan. In alternative embodiments, the treatment plan including power settings can be created as a combination of automatic and user input selection of treatment parameters to carry out the ablation autonomously.

Optionally, at a process 355, one or more indicators of the target tissue, the treatment locations, the power settings, the ablation pattern, the landmarks, and/or the structures to treat or avoid are displayed on, included within, or overlaid on, the updated model generated in process 340 to create an updated planning model. The planning anatomic model and treatment plan can be displayed as instructions on a display, as indicators overlaid on the anatomic model generated during process 340, and/or as updates to the anatomic model. For example, using display system 110 shown in FIG. 1, visual instructions can be provided indicating location of target tissue, power levels for ablation, duration of ablation, warning of proximity to sensitive anatomy, etc. Instructions can be audible, visible, or haptically provided. Indicators can be overlaid on the anatomic model to show location and depth of diseased tissue or to indicate location and depth of ablation lesions (lesions created within tissue after applying ablative energy). The instructions and/or indicators can be highlighted within the current updated model by being displayed in a different color, transparency, hue, size, etc.

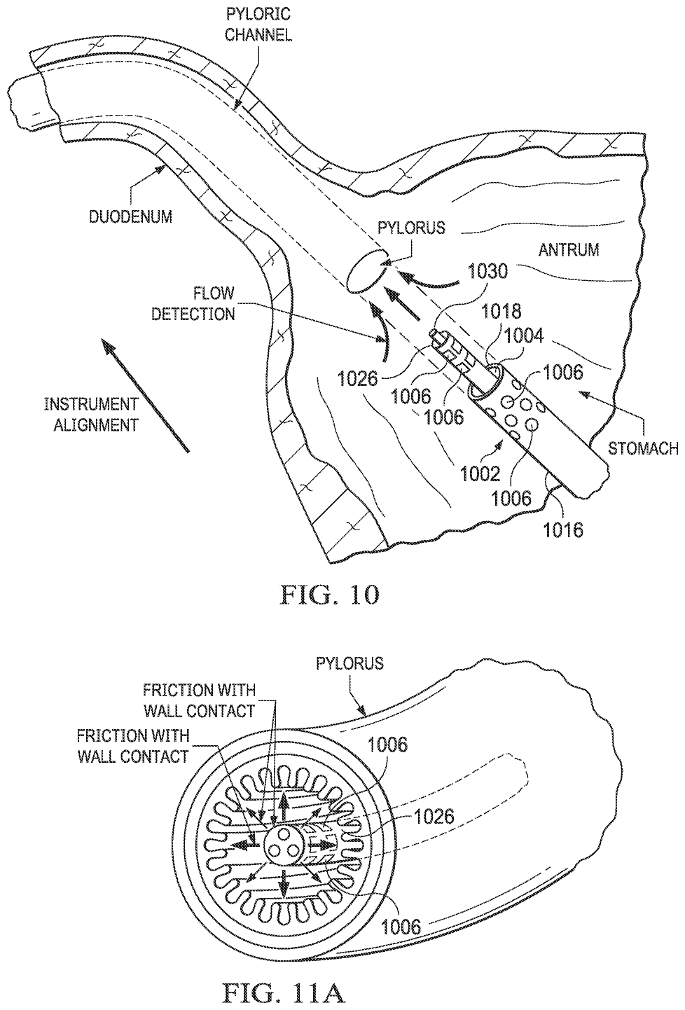

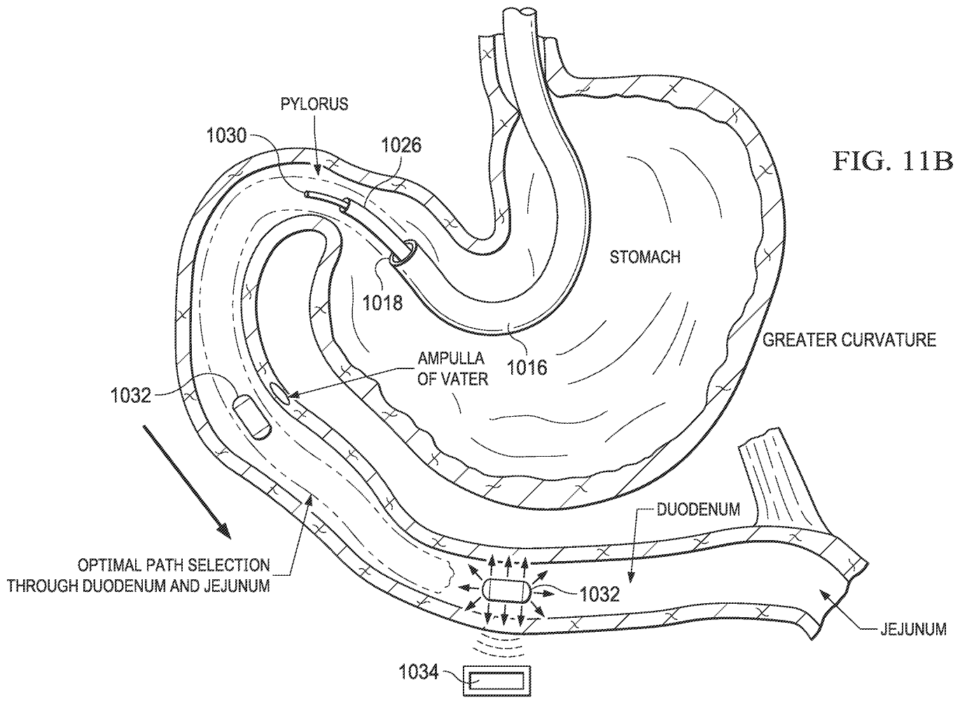

A treatment/diagnostic procedure can be performed in process 360 by using the procedure plan determined in process 350. Real time navigational guidance optionally may be displayed within the updated anatomic model. The navigational guidance optionally may include a rendered image of the medical device represented within the anatomic model. In order to display the rendered image of the medical device in the anatomic model correlated to a real time pose of the actual device within the patient anatomy, the device should be registered to the anatomic model. In some embodiments, the model is built from data, information, fiducial markers or images collected using the visualization device so that the model is inherently registered to the device, since the visualization device (or medical device coupled to the visualization device) includes localization sensors. In some embodiments, the model is built from data collected during insertion of the device within anatomy, where data points measured using localization, e.g., shape sensing, are used to build the model. Therefore, the model and the device are inherently registered. In some embodiments, an initial model is generated from pre-operative data, e.g., data obtained from a capsule endoscope or capsule sensor 1032 (FIG. 11B) or delivery of nanoparticles and imaging agents directly into the gastrointestinal wall, and the device is navigated through anatomy, collecting positional data points, and identifying target areas of interest, e.g., identifying landmarks using a camera. The collection of the target area such as the landmark can also be correlated with a position or orientation of the camera. Therefore, a system can use the positional data and identified landmarks to register the device to the initial model. Examples of such systems are disclosed in detail in U.S. patent application Ser. No. 13/107,562 (filed May 13, 2011)(disclosing “Medical System Providing Dynamic Registration of a Model of an Anatomic Structure for Image-Guided Surgery”) and International Patent Application Number PCT/US2016/033596 (filed May 20, 2016)(disclosing “Systems and Methods of Registration for Image Guided Surgery”), which are incorporated by reference herein in their entirety.

Once registered, the rendered image of the elongate device or medical device can be updated as the operator navigates the device to various points in anatomy. The navigational guidance can include the treatment plan, which optionally includes treatment parameters determined during process 350, such as indicators showing locations, sizes, depth, number, pattern, and number of treatment zones within the model. In some embodiments, the indicators can be altered in color, transparency, hue, size, etc., as ablative energy is delivered to tissue, thus providing the user with visual feedback of energy delivery. In some embodiments, the change in appearance of the indicators can be based on time of energy delivery and/or power of energy delivery. In other embodiments, the change in appearance of the indicators can be based on an expected effectiveness of ablation based on duration of energy delivery, power, depth and tissue type. In some embodiments, impedance sensors integrated into an ablation probe can measure impedance of tissue and be used as a measure of ablation effectiveness which can be reflected by a change in appearance of the indicators. The model can indicate the location and depth of target tissue, indicators (such as arrows or lines) can be displayed to guide the user from a current position of the device to the target tissue, an indicator can alert the operator to begin ablation when the device is positioned at target tissue, power levels and power durations can be displayed and adjusted as tissue becomes more desiccated during ablation, instructions can be provided to apply more pressure to tissue for deeper ablation, larger or smaller surface areas, etc. The model can visually update an appearance of diseased tissue as it becomes ablated by changing color, shade, transparency, or hue relative to healthy or non-ablated tissue.

In one embodiment, the medical procedure plan can provide for display of an indicator that instructs deployment of a protective device or a protective substance prior to delivering ablative energy or treatment. In one example, a protective device or protective cooling substance is deployed through a working channel of the elongate instrument and at the position determined during process 350. Navigational guidance can be provided to aid the operator in positioning of the elongate device during delivery, positioning, and deployment of the protective device.

The procedure plan may be executed in a manual, semi-manual/partially-automated, or fully automated/autonomous manner according to the navigational guidance and treatment plan. For example, the ablation probe may be manually positioned by the operator, but as the registered and localized ablation device is positioned within anatomy, the medical instrument system can automatically apply power at a power level, frequency and a duration as provided by the treatment plan when the ablation device is positioned at target tissue. In some embodiments, power may be automatically reduced or turned off if the system detects the ablation probe is close to sensitive areas. In some embodiments, if a sensitive area is located in only a portion of the circumference of a target area (e.g., within a body lumen), then power is delivered only to the opposite side of the target area to avoid the sensitive area. Endoscopic visualization may be used so that the user may stop the ablation and/or override the automated procedure. In some embodiments, image-based recognition may be used to automatically identify when the ablation device has reached target tissue, when ablation is completed, or if the ablation probe is too close to sensitive areas.

The processes shown in the flowchart of medical procedure 300 may include additional processes or be performed in any order needed. For example, in some embodiments, target areas of interest may be identified in process 330 before the model of anatomy is created in process 320. In some embodiments, in process 320 an initial model is made from a generic model of human anatomy or from data such as a CT scan, then the target areas of interest in process 330 are identified during the real time procedure as the anatomy is explored, and then the initial model is updated in a separate process. In some embodiments, the model in process 320 is made while identifying target areas of interest in process 330. Additionally, in some embodiments, additional processes are performed. In some embodiments, the medical procedure performed is used to update the model. For example, the model may be updated with locations of ablated, partially ablated or non-ablated target tissue. Thus after an initial treatment performed in process 360, the procedure can return to process 340 in which the location, size, and depth of ablated, partially ablated or non-ablated tissue is displayed on the current anatomical model to create an updated model, which can be used to create an updated treatment plan requiring additional ablations.

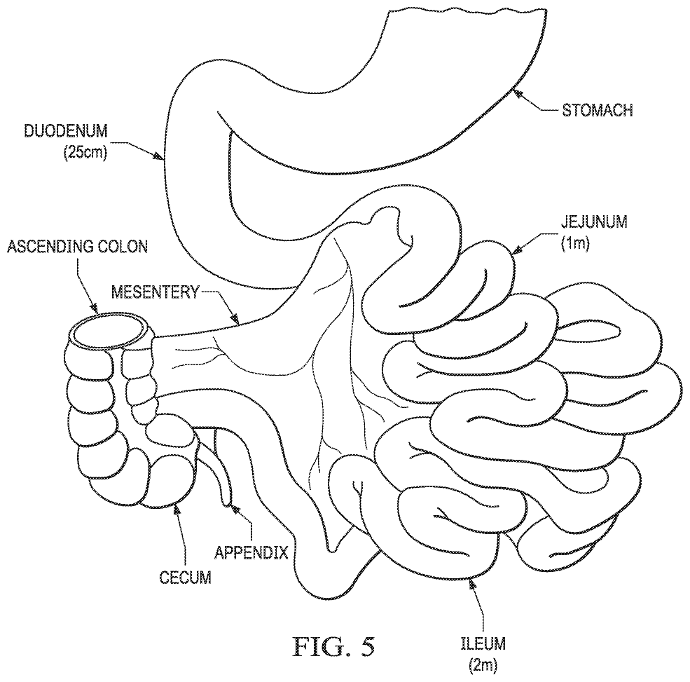

In one example, the systems and methods described above with regard to medical procedure 300 can be applied to mapping and treatment of a patient's gastrointestinal tract. FIG. 5 illustrates a representation of a portion of a patient's digestive system, including the stomach which leads to the small intestine. The stomach contains cells known as the Myenteric interstitial cells of Cajal (ICC-MY) which serve as a pacemaker which create the bioelectrical slow wave potential that leads to contraction of the smooth muscle in the upper GI tract. The small intestine includes three sections—the duodenum, jejunum, and ileum. The duodenum includes several layers of tissue, which include an innermost mucosal layer, which covers a submucosal layer, and muscle layers found deeper within the duodental wall. Cells within the mucosal layer of the duodenum produce one or more hormones that impact insulin production. When the mucosal cells become hyperplastic, an overabundance of hormones is produced, which affects insulin secretion. The resulting impact on insulin secretion can result in medical conditions known as insulin resistance and Type II diabetes. It is possible to treat the cells of cajal in the stomach, enteric nerve plexus in the proximal intestines and hyperplastic mucosal cells within the small intestine to treat such conditions. For example, all or a portion of the duodenal and jejunal mucosal tissue can be ablated to slough off the hyperplastic mucosal cells. After ablation, the submucosa can generate replacement mucosal cells unaffected by hyperplasia, thereby restoring normal insulin secretion and body response.

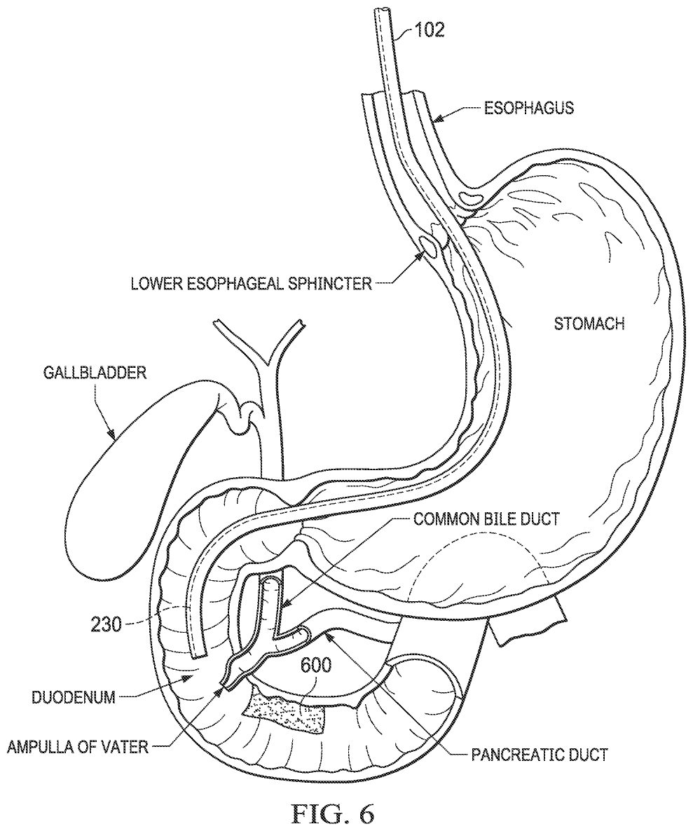

In some current medical procedures, a balloon catheter ablation device is endoluminally delivered over a guide wire via a patient esophagus and stomach into the duodenum for ablation of hyperplastic, neoplastic, dysplastic, diseased, or other tissue along a length of the duodenum. Yet current procedures suffer drawbacks and challenges. Because the lining of internal passageways and hollow viscus structures are soft and distensible, areas such as the lining of the esophagus, stomach, and small intestine (e.g., the duodenum, biliary tree, pancreatic ducts, colon, and rectum can be damaged or perforated by the tip of the guide wire or by an instrument. Likewise, similar treatment in other body areas, such as the sinuses (e.g. front, maxillary, sphenoid, and ethmoid)) suffer drawbacks and challenges. Additionally, positioning of the device near target tissue is commonly performed under fluoroscopy, which often does not display hyperplastic tissue so that the ablation is performed blind while at the same time exposing the clinical operator and patient to radiation. While some procedures can be performed under endoscopic visualization where the hyperplastic tissue can be visible, the operator must avoid areas of sensitivity, such as areas near the union of the pancreatic duct and the common bile duct, e.g., the ampulla of Vater (hepatopancreatic ampulla) and the papilla of Vater (major duodenal papilla) as can be seen in FIG. 6, which can be difficult to visualize, especially when endoscopic views are compromised by debris.

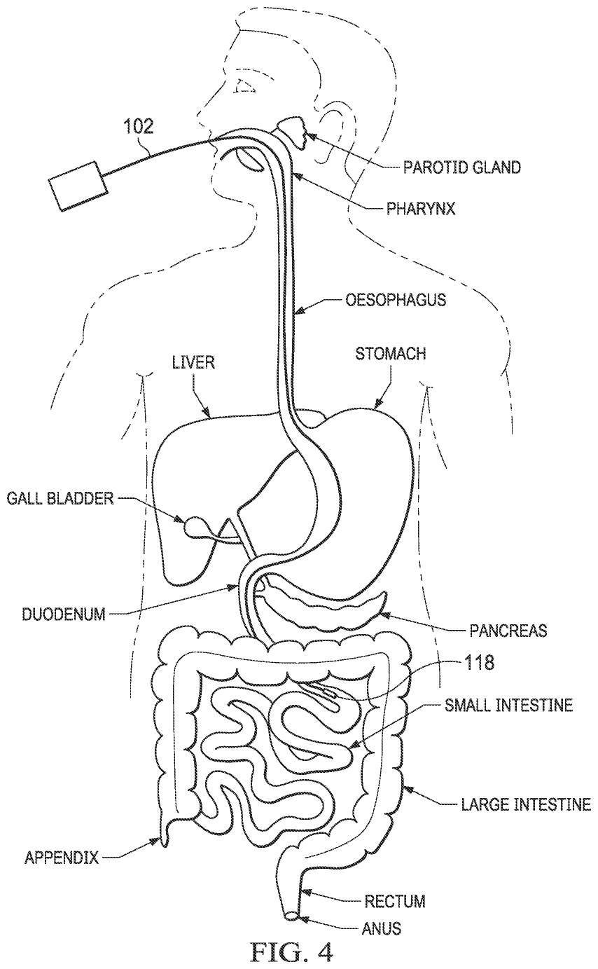

Referring to FIG. 3 and FIG. 4, an example of applying medical procedure 300 to a patient digestive system is provided, including generating a model of the gastrointestinal tract of a patient anatomy. The model may be used to plan areas of treatment, and/or used for treatment of hyperplastic tissue within the duodenum. For example, the elongate device 102 can be used to access and create a model of portions of the small intestine such as the duodenum, the jejunum, and/or the ileum. In one example, the elongate device 102 can be navigated to the duodenum and/or jejunum as illustrated in FIG. 4. At process 310, the elongate device 102 can be inserted into a patient's mouth and navigated through the patient digestive tract including through the esophagus, through the gastroesophageal junction, through the stomach, and through the small intestine to an anatomic target. In an alternative embodiment (not shown), an external surgical incision could be made to provide direct access to the stomach and the elongate device can be inserted into the duodenum and/or jejunum from the stomach. The more direct access approach directly from the stomach, would allow for shorter elongate devices to be used during a procedure.

Referring to process 320, an anatomic model, or map of the gastro-intestinal tract can be created. As previously described, the gastro-intestinal model may be generated from data collected using localization sensors (e.g., fiber optic shape and/or a plurality of EM sensors) coupled to the elongate device 102, as the elongated device 102 is navigated through the digestive tract, and well as information from imaging sensor 230 disposed through elongate device 102 or near a distal end of elongate device 102. In some embodiments, a processor such as control system 122 may collect position and/or localization data representing the distal end 118 of the elongate device 102, thereby providing a centerline of the gastrointestinal tract. In some embodiments, the centerline represents a path through the esophagus, through the stomach, and into the small intestine. In some embodiments, once the elongate device 102 is navigated to the target hyperplastic tissue, shape sensor data measuring the full shape of the elongated device 102 is utilized to generate an instant centerline of the full gastrointestinal tract or to supplement the centerline model generated during collection of points representing the distal end 118 of the elongate device 102. The model may then be built to include diameters of the esophagus, walls of the stomach, walls of the duodenum and walls of the jejunum by using either standard anatomical human data or by using elongate device 102 and localization sensing to touch anatomical walls to determine volume, and/or data from imaging sensor 230 where diameter can be determined using image-based methods.

FIGS. 5-6 illustrate a representation of patient anatomy including the esophagus, stomach, duodenum, and jejunum. Within the duodenum, the papilla of Vater forms a protrusion adjacent the ampulla of Vater, which is illustrated connecting the pancreatic duct and the common bile duct. As shown in FIG. 6, hyperplastic tissue 600 can be located near structures such as the papilla of Vater/ampulla of Vater. During ablation of tissue, damage to the papilla of Vater and ampulla of Vater can be detrimental to patient health, causing conditions such as pancreatitis. Thus it is important to identify structures as sensitive anatomy to avoid during ablative treatment, such as the ampulla of Vater. Referring again to process 330, therefore, identifying target areas of interest can include identifying anatomical target areas, such as areas identified for treatment, such as hyperplastic tissue; identifying structures or anatomy to be avoided during treatment, such as the ampulla of Vater (as shown in FIG. 6), which may be located in close proximity to the treatment areas; and/or identifying artificial locations, such as a previously placed real or synthetic markers within the tissue. Hyperplastic tissue, hypertrophied tissue, nerves, entero-endocrine cells, goblet cells, cells of cajal or other target tissue and various anatomical structures (papilla of Vater, ampulla of Vater, etc.) can be identified visually in endoscopic camera images as well as in, e.g., ultrasound or OCT images or scanned OCT data formulated into images. In some embodiments, such landmarks can be user-identified. The clinical operator may signal the location of the landmark by pressing a button when the elongate device is adjacent to or touching a landmark. In some embodiments, ultrasound may be used to determine depth of hyperplastic or hypertrophic tissue. With localization sensors provided within the elongate device, an exact location of the hyperplastic tissue, hypertrophic tissue, nerves, entero-endocrine cells, goblet cells, cells of cajal or other tissue correlated to depth of the tissue can be determined.

Referring back to process 340, the target areas of interest identified in process 330 may then be displayed or rendered on the model of the anatomy generated in process 320 as previously described. The target areas of interest can include hyperplastic or hypetrophic tissue, nerves, entero-endocrine cells, goblet cells, cells of cajal (stomach), the papilla of Vater, and the ampulla of Vater. In some embodiments, if ultrasound is used to identify depth of hyperplastic tissue, then the hyperplastic tissue can be displayed at a measured location within the model, at a detected depth based on ultrasound data, and in a different color, shade, hue, or transparency from normal tissue.

Referring to process 350, the model may be used to plan a medical procedure for treatment of hyperplastic tissue, hypertrophic tissue, nerves, entero-endocrine cells, goblet cells, cells of cajal (stomach) or abnormal tissue, including determining a navigational path to the hyperplastic tissue and/or creating a treatment plan. The navigational path can be established within the model by the user by manually identifying a path through a patient's mouth, through the esophagus, through the gastroesophageal junction, through the stomach to identify the pacemaker cells of cajal, through the pyloric sphincter, and into the duodenum and jejunal portion of the small intestine. In other embodiments, the computer processor can automatically generate a similar path through anatomy to the hyperplastic tissue within the small intestine. As previously described, the treatment plan can be automatically or manually created to identify treatment parameters such as treatment location, size of treatment area, treatment depth, number of treatments, and spacing of treatments. Sensitive areas to avoid within anatomy such as the papilla of Vater/ampulla of Vater can be automatically or manually identified, and power levels can be automatically or manually set and saved within the procedure plan to reduce power near the sensitive anatomy and to increase power levels at locations with hyperplastic tissue. Additionally the sensitive areas can be identified as locations of deployment for protective devices and/or protective substances.

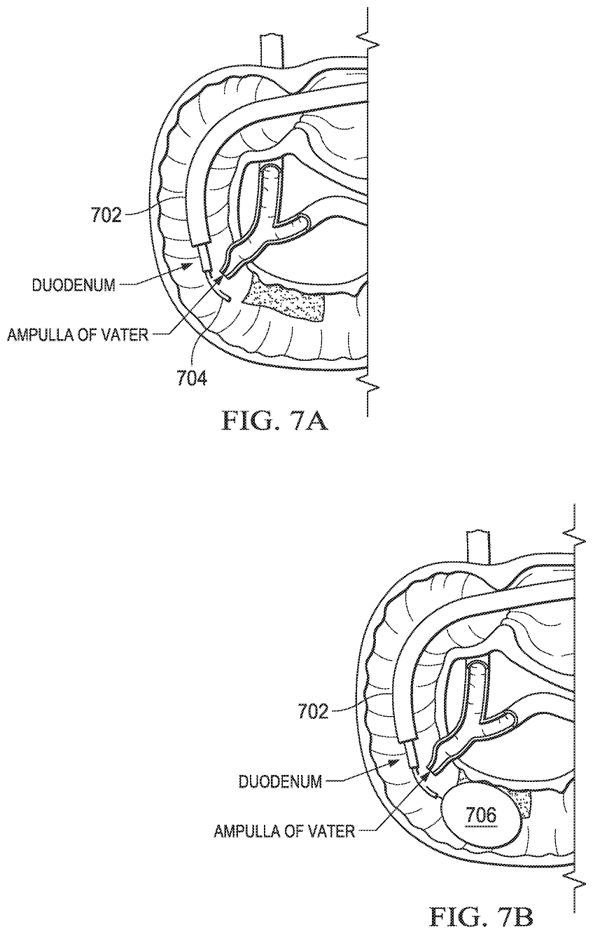

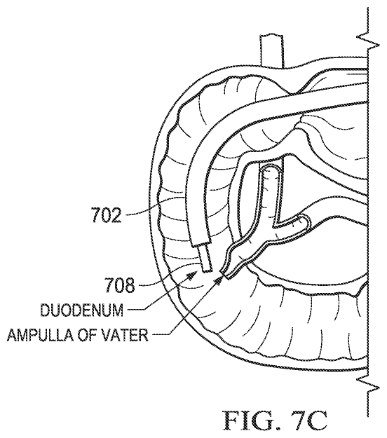

Using the medical procedure plan determined in process 350, a treatment procedure for hyperplastic tissue, hypertrophic tissue, gastrointestinal (enteric) nerves, cells of cajal in the stomach or other abnormal tissue can be performed in process 360. FIGS. 7A through 7C illustrate a portion of a patient small intestine including the duodenum, the papilla of Vater/ampulla of Vater. and hyperplastic target tissue. In one example, the treatment procedure of process 360 begins with an elongated device 702 (such as device 102, 202) positioned within the target anatomy as illustrated in FIG. 7A such that the elongate device 702 is positioned near the target hyperplastic or hypertrophic tissue. The elongate device can include a working lumen for delivery of tools and devices required for the medical procedure. In some examples, the medical procedure plan includes deploying a protective device or protective substance. For example, the working lumen, or a separate delivery device can be deployed through the working lumen, may be used to deliver saline or water to cool areas surrounding the target ablation area. In another example, a sheath 704 can be deployed through the working lumen of the elongate device, over the papilla of Vater and ampulla of Vater. The treatment device can then be deployed through that sheath, protecting the papilla of Vater and ampulla of Vater when treatment energy is delivered.

Methods of treatment can include expanding the submucosal layer such as by fluid injection to flatten it and using a balloon 706 filled with hot air, water or a combination thereof to ablate the tissue as illustrated in FIG. 7B. The contact balloon treatment could also be performed by using radio frequency (RF), ultrasound, or microwave with a fluid filled balloon. A second class of treatments (not shown) rely on plugging a section of duodenum and or jejunum to be treated and then applying the treatment methodology, such as flowing gas, heated vapor, or applying chemical such as alcohol. A third class of treatments are more targeted ablation therapies, including RF, microwave, ultrasound, direct heat, laser ablation, ultra-sonic waves, plasma energy, electroporation, cryoablation, etc. delivered by an ablative medical instrument 708 as shown in FIG. 7C may be used to target specific areas of hyperplastic or hypertrophic tissue. Such treatments as laser and sprays may be used for shallow penetration, thus removing the need to protect layers under the mucosal layer by injection of fluid first into the submucosal layer. Finally, mechanical devices, such as a hydrojet, physical scraper or a resecting device, could be used.

The treatment plan can include not only locations, surface area, sizes, and depths of hyperplastic and hypertrophic tissue but also margins around hyperplastic tissue at a lesser depth than where the hyperplastic and/or hypertrophic tissue was visibly initially identified during the identification stage. During the delivery of ablative energy, hyperplastic, hypertrophic, abnormal or other target tissue and various anatomical structures can be identified visually in endoscopic camera images as well as in, e.g., ultrasound or OCT images. Tissue may be identified as ablated, partially ablated, or non-ablated tissue by measuring impedance of the tissue and monitoring a change in impedance or comparing impedance measurements against pre-determined impedance thresholds. The operator can deviate from the medical plan saved during process 350 and can also deviate from location and power delivery based on live endoscopic images. In some embodiments, power can automatically be reduced and or turned off based on proximity to sensitive areas established within the medical plan. In some embodiments, if a sensitive area is disposed in only a portion of the circumference of a target area, such as the papilla of Vater and ampulla of Vater, then power can be delivered only to the opposite side of the target area to avoid the sensitive area. Optionally, algorithms utilizing thermal modeling, for example, from CEM43 can be utilized to optimize energy delivery.

In some examples of medical procedures, within a body lumen such as the gastrointestinal tract, the medical device may be used as a diagnostic or treatment device for insulin resistance as will be described in detail below. In another example, the medical device may be routed to the esophagus to ablate various diseases, such as esophageal cancer. In another example, the medical device can be routed through a patient trachea into branches of patient airways to model patient lungs for mucosal resurfacing or ablation for treatment of lung conditions. Individual airway branches can be marked visually during navigation and treatment overlaps can be determined during a procedure such as for treatments for chronic obstructive bronchitis or asthma (for example, using RF ablation, cryospray, plasma energy, ultrasonic waves, microwave, electroporation, etc.). In yet another example, the medical device can be used for urology cases to real time map the chambers of the kidney. Initially, the medical device can be used to explore all the chambers of the kidney, including explorations to endoscopically visually determine the location and size of kidney stones. A model of the kidneys can then be created to include mapping of the location and size of the kidney stones. The medical procedure can then include using the medical device, or delivering a tool through the medical device, to break the kidney stones manually or with the assistance of a holmium laser. After stone breakage, the user can deliver the medical device to the different calyxes to visually examine stone fragments remaining from the stone breakage procedure. The location and size of the remaining stone fragments can then be used to update the model of the kidney and enables all of the kidney chambers and remaining stones to be tracked. Mapping and modeling is particularly useful because not all patients with kidney stones have a CT taken, and not all patients with kidney stones have a pre-operative image/map. Thus the map created during the medical procedure could be the only navigational guide provided to a user.

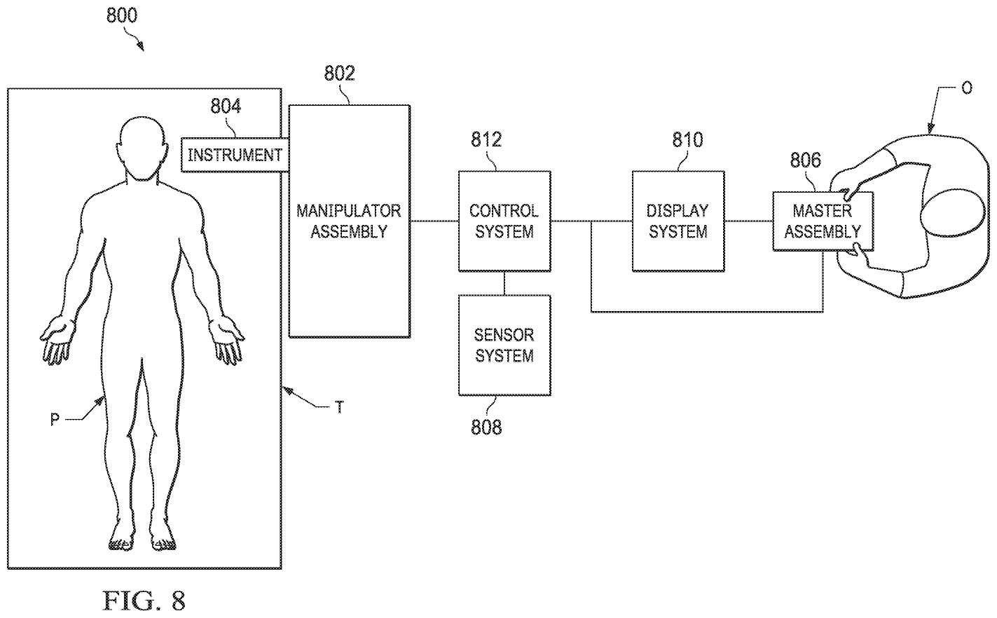

In some embodiments, the medical instrument 226 may be an ablation probe and may be controlled at least in part with computer assistance so an ablation pattern may be automatically or autonomously created according to the treatment plan. In some embodiments, the navigation of the ablation probe can be fully automated from first entry into patient anatomy, during navigation of the ablation probe to target tissue, and during ablation according to the navigation and treatment plans. FIG. 8 is a simplified diagram of a computer-controlled teleoperated medical system 800 that may be used to control instruments 804, such as ablation probes, medical instruments, elongate devices, and/or the like. In some embodiments, teleoperated medical system 800 may be suitable for use in diagnostic and therapeutic procedures described herein.

As shown in FIG. 8, medical system 800 generally includes a manipulator assembly 802 for operating a medical instrument 804 in performing various procedures on a patient P. The manipulator assembly 802 may be teleoperated with at least partial computer assistance, manually operated, or operated as a hybrid computer and manual assembly with select degrees of freedom of motion that may be motorized and/or select degrees of freedom of motion that may be manually operated. A master assembly 806 allows an operator O (e.g., a clinical user as illustrated in FIG. 8) to control manipulator assembly 802. A display system 810 (e.g., the display system 110) allows the operator to view the treatment site by displaying a live image and/or a representation of the treatment site and medical instrument 804 generated by subsystems of sensor system 808, as well as displaying images and instructions for navigational guidance. Sensor system 808 can include one or more subsystems for receiving information about the instruments of manipulator assembly 802. The sensor system 808 may include sensors 106, 108, for example. Such subsystems may include a position/location sensor system (e.g., an EM sensor system); a shape sensor system for determining the position, orientation, speed, velocity, pose, and/or shape of a distal end and/or of one or more segments along a flexible body that may make up medical instrument 804; and/or a visualization system for capturing images from the distal end of medical instrument 804. Teleoperated medical system 800 may also include control system 812 (e.g., the control system 122). Control system 812 includes at least one memory and at least one computer processor (not shown) for effecting control between medical instrument 804, master assembly 806, sensor system 808, and display system 810. Control system 812 also includes programmed instructions (e.g., a non-transitory machine-readable medium storing the instructions) to implement some or all of the methods described in accordance with aspects disclosed herein, including instructions for providing information to display system 810. In some embodiments, control system 812 may receive force and/or torque feedback from force sensors in the medical instrument 804.

Aspects of the systems and methods described in the present disclosure include (i) navigation and control of medical devices to one or more target areas; (ii) visualization and mapping of the one or more target areas and real-time tissue-characterization in the target areas; (iii) selective treatment within target areas while protecting sensitive areas; and (iv) tracking and visualization of the applied treatment and area of anatomy treated, and optionally measurement and/or characterization of treatment effectiveness. By way of example, the system and methods can be applied to a patient's digestive system, such as treatment in the stomach, duodenum, and/or jejunum for the treatment of diabetes and other metabolic diseases. While some embodiments are provided herein with respect to the stomach, duodenum, and/or jejunum, the systems and methods described are not limited to treatment in these areas, and the present disclosure may be directed to treatment in other body lumens, such as in other portions of the digestive system (esophagus, rectum, colon), or in other enclosed spaces within the body.

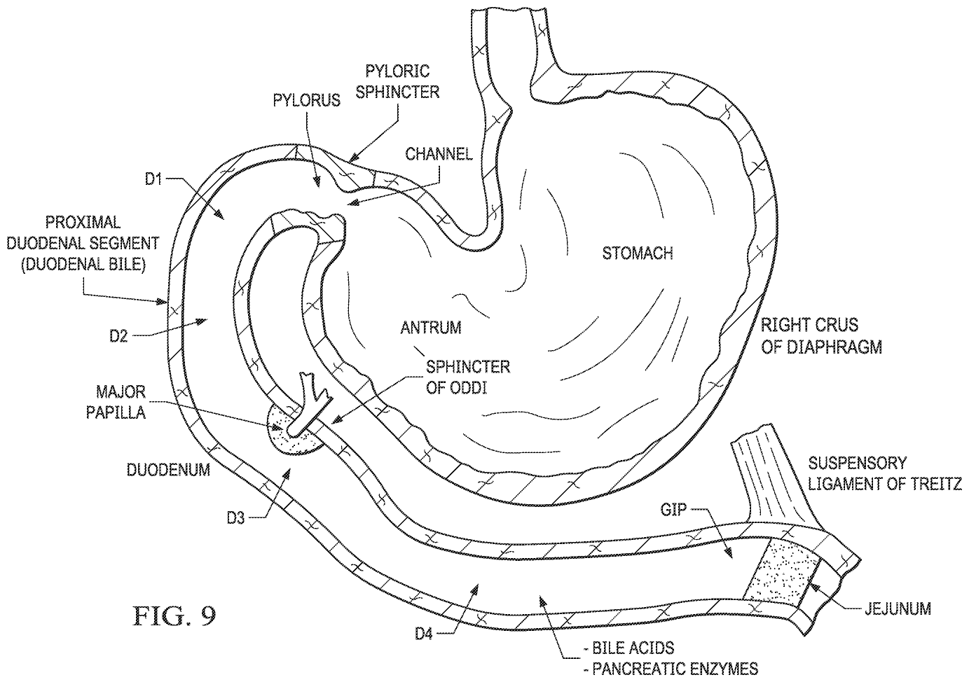

As described above, selective removal (e.g., via ablation) of the cells of cajal within the stomach, and hyperplastic entero-endocrine mucosal cells, hypertrophic mucosal cells and enteric nerves within the duodenum and/or jejunum can be used to treat insulin resistance related to Type II diabetes in order to restore normal insulin secretion. Some or all of the mucosal tissue layer of the duodenum and/or the jejunum can be removed to eliminate the hyperplastic and hypertrophic mucosal cells and enteric nerves, which the body then over time replaces with mucosal cells unaffected by the entero-endocrine hyperplasia. The long-term benefit of such a treatment is dose-dependent and is impacted by the amount of tissue surface area that is treated. FIG. 9 illustrates potentially problematic areas that may be encountered with navigation and treatment in the duodenum. Treating a greater surface area of the duodenum and/or jejunum mucosal lining along the circumference and the length of the lumens is correlated with better outcomes. To that end, treatment may be improved by selective removal of the mucosal layer in the proximal region of the duodenum (regions D1 and D2 in FIG. 9, which are proximal to the ampulla of Vater), by selective removal of the mucosal layer in the distal region of the duodenum (region D4 in FIG. 9, which is distal to the ampulla of Vater), and/or by selective removal of the mucosal layer in at least the proximal region of the jejunum. In some embodiments, treatment may be provided by selective removal of portions of the mucosal layer in the duodenum alone. In other embodiments, treatment may be provided by selective removal of portions of the mucosal layer in the jejunum alone. And in yet other embodiments, treatment may be provided by selective removal of portions of the mucosal layer in at least a portion of each of the duodenum and the jejunum. For treatment, the mucosal layer has a superficial portion and a deep portion. Stem cells are present in the deep portion and in the underlying submucosal layer, and removal of a majority of the cells within the mucosal and submucosal layers will leave a small portion of stem cells able to regenerate the mucosal lining of the gut. The duodenum is approximately 25-40 cm long, 25 mm in diameter, and the duodenum wall is approximately 2-3 mm thick. Accordingly, removal of sub-millimeter to millimeter depth ranges of the inner side of the luminal wall may be used to achieve adequate treatment. Portions of the submucosal layer may also be treated and removed, but the underlying muscle layer should be minimized, avoided or not be reached or compromised to avoid potentially harmful trauma.

In addition, the submucosal layer of the duodenum contains a bundle of nerves known as the submucosal plexus and enteric nerves. In some embodiments, portions of the submucosal plexus may be targeted and removed to restore insulin function, as described in further detail below.

The present disclosure describes aspects for navigation and control of a teleoperated treatment device, for example inserted into the mouth and through the digestive tract for treatment for treatment in the duodenum/or and the jejunum. The teleoperated treatment device may be implemented as part of the teleoperated medical system 800, described above, and controlled using control system 122/812. The described aspects overcome several challenges associated with navigation and control of manually controlled treatment devices.

Aligning and Navigating from the Stomach Through the Pyloric Sphincter and into the Pyloric Channel

Navigation from the stomach through the pyloric sphincter and into the duodenum provides an example application of alignment to navigate through an internal body lumen restriction. When a manually controlled treatment device is manually navigated into the stomach, it may be difficult to properly align the device's distal tip with the pyloric sphincter opening in order to insert the tip through the pyloric sphincter and into the duodenum. Not only must the pyloric sphincter be identified, the endoscope's distal end must be aligned with the first portion of the duodenum so the endoscope passes easily into the duodenum avoiding damage to the surrounding lining of the gut. It may also be difficult to visualize the pyloric sphincter opening because of the large hollow viscous nature of the stomach with multiple rugae collapsing around the opening of the pyloric channel. Endoscopists have developed numerous manual techniques to overcome these challenges. One technique includes looping the flexible treatment device in a 360-degree loop in the stomach to align the distal tip of the device with the pyloric sphincter, and then manipulating the device to remove the loop after the tip has passed through the pylorus. But, this looping is undesirable because it may cause discomfort to the patient despite light to moderate sedation. Such looping may also cause additional friction on the treatment device as the device is advanced, so that the device is not reliably advanced in a constant 1:1 proximal push:distal advance ratio when traversing patient anatomy. The indefinite push:advance ratio complicates the navigation procedure. Insufflation is also used to flatten out the pyloric antrum sidewall in order to better visualize the pylorus. Endoscopists often require lengthy training and practice in order to learn to effectively manually navigate a duodenal endoscope through the pyloric channel and into the duodenum for observation and treatment.

In some embodiments, computer-assisted teleoperated systems and methods provide improved alignment and navigation for difficult-to-traverse pathways, such as from the stomach, through the pyloric sphincter and pyloric channel, and into and through the duodenum. FIGS. 10 and 11B illustrate simplified diagrams of an elongate device 1002 located in the stomach near the pylorus. Elongate device 1002 is similar in construction and function as elongate devices 102 and 202 described above, except where described herein. Elongate device 1002 includes a flexible body 1016 having a distal end portion 1018, main lumen 1004, one or more instruments 1026 (e.g., catheters 1022) in the main lumen, one or more navigation sensors 1006, and one or more imaging sensors 1030. The elongate device 1002 may be implemented as part of the computer-assisted teleoperated medical system 800, described above, and controlled using control system 122/812. The various imaging sensors 1030, navigation sensors 1006, and imaging analyses described herein may be used in isolation or in combination to assist with manually or automatically navigating the elongate device 1002 through the pylorus and into the pyloric channel using the computer-assisted teleoperated medical system 800, as feedback to align and navigate the elongate device 1002 with the pyloric sphincter and pyloric channel. For example, via the computer-assisted teleoperated medical system 800, the pose of the distal end portion 1018 relative to the pylorus may be adjusted manually or automatically based on the feedback from the sensors and imaging analyses.

The one or more imaging sensors 1030 may be substantially similar to imaging sensor 230 described above, and optionally they may be included within any type of minimally invasive tool for providing intraoperative images. In various embodiments, imaging sensors 1030 may be slideably disposed through main lumen 1004, slideably disposed within a secondary lumen offset from main lumen 1004, fixed within the main lumen or the secondary lumen, integrated into a wall of the elongate device 1002, fixed to the instruments 1026, or disposed external to elongate device 1002. The one or more imaging sensors 1030 may include stereoscopic or monoscopic endoscopic cameras, endoluminal or intravascular ultrasound devices, a fiber-optic bundle such fiberscope, optical coherence tomography (OCT) devices, and/or the like. Imaging sensors 1030 may include any combination of any number of endoscopic, OCT, and/or ultrasound probes. As with imaging sensor 230, imaging sensors 1030 may include a distal portion at or near the distal end portion 1018 of the flexible body 1016 for capturing images and a cable coupled to the imaging sensors 1030 for transmitting the captured image data. The captured image data is processed by an imaging system, such as visualization system 131, for display and/or for use in a tracking system, such as tracking system 130, to support tracking of distal end portion 1018 and/or a portion of flexible body 1016 along the length of flexible body.

The one or more navigation sensors 1006 are used to assist with aligning the distal end of the treatment device with the pyloric sphincter. Thus, in some examples, the navigation sensors 1006 may be referred to as alignment sensors. The navigation sensors 1006 may include one or more sensors located at or near the distal end portion 1018 of the elongate device 1002. The navigation sensors 1006 may further include one or more sensors located proximal to the distal end portion 1018. In some embodiments, the navigation sensors 1006 include either a single sensor or a plurality of sensors distributed along the length of the elongate device 1002 (e.g., along the length of the flexible body 1016). In some embodiments, the one or more navigation sensors 1006 are fixed to the instruments 1026. The navigation sensors 1006 are used to capture navigation sensor information, which is then provided to a tracking system, such as tracking system 130, to support tracking of distal end portion 1018 and/or a portion of flexible body 1016 along the length of flexible body. In some embodiments, the imaging sensors 1030 and the navigation sensors 1006 are used to determine, for example, one or more of the position, orientation, pose, shape, speed, and/or velocity of distal end portion 1018 and/or of one or more lengths along the flexible body 1016.

The navigation sensors 1006 may include one or more flow sensors to determine fluid (liquid, gas, or both liquid and gas) flow direction through the pylorus in order to aid with alignment of the elongate device 1002 for easy passage through the pyloric sphincter and into the duodenum via the pyloric channel for tracking (e.g., track velocity of stomach outgoing flow through the pyloric sphincter). Such fluid or gas flow sensors may include one or more of ultrasonic, magnetic, mass, vortex, thermal, and/or ultrasound sensors, or any combination thereof. The navigation sensors 1006 may be used to track fluid or gas flow such that the user can orient the distal end portion 1018 towards the direction of the fluid flow to identify and traverse the pyloric sphincter. Fluid and gas flow gradients may be used to redirect the distal end portion 1018 towards the direction of maximum flow, which may be aligned with the center of the pyloric channel. In some examples, the one or more navigation sensors 1006 may also include one or more acoustic sensors.

The navigation sensors 1006 may further include strain sensors, including capacitive strain sensors, or other force transducers to provide feedback to user on a path of least friction to insert the device 1002 through a lumen, such as insertion into the pylorus (see, e.g., FIG. 11A). As described in further detail below, the navigation sensors 1006 may additionally or alternatively be used to provide feedback to user on a path of least friction to traverse the device 1002 through the duodenum and/or jejunum. The strain sensors may be located at or near the distal end portion 1018 of the elongate device 1002, at one or more proximal portions of the device 1002, or both. Such strain sensors may include one or more electrical (e.g., capacitive) strain gauges, optical (e.g., optical Fiber Bragg Grating) strain gauges, and semiconductor strain gauges, or a combination thereof. The strain sensors may be distributed around the circumference of the device 1002 to detect a circumferential direction of strain. In some embodiments, the strain sensors may be used to sense strain information that is used to provide tactile feedback to a user, with larger feedback values indicating a possible misalignment between the distal end portion 1018 and the pylorus. Via the computer-assisted teleoperated medical system 800, the pose of the distal end portion 1018 relative to the pylorus may be adjusted manually, automatically, or under robotically assisted control to reduce the sensed forces and thus reduce friction on the device 1002, for example, as the device 1002 navigates the pylorus and into the duodenum. Further, in some embodiments, as described in more detail below, the pose of the distal end portion 1018 may be adjusted manually or automatically to reduce the sensed forces, for example, as the device 1002 travels through duodenum. In some embodiments, for example where treatment in the jejunum is targeted, such feedback may also be used to manually or automatically adjust the pose of the distal end portion 1018 as the device 1002 navigates past the ligament of treitz where the duodenum transitions into the jejunum. In some embodiments, the direction of contact between the device 1002 and a body wall may be detected and automatically corrected, for example, to move the device 1002 in a direction away from the contact (e.g., by moving the device 1002 a distance apart from the contact) or navigational feedback can be provided (e.g. visually, audibly, haptically) to provide guidance for correction. The distance of movement may be based on the magnitude of sensed strain feedback. As described in further detail below, in some embodiments, a re-centering mechanism may be used to manually, robotically assisted with navigational guidance, or automatically center the device 1002 in a lumen, and in further embodiments, portions of the device 1002 may be rigidized to minimize drag or contact with the wall of the lumen. Such rigidization may be independent of the centering or may be used in conjunction with the centering. In some embodiments in which the device tip has selective compliance, strain may be measured by software/firmware/hardware as the user advances the device. In some embodiments, impedance- or admittance-based control may be implemented to use strain (or equivalently force/torque) or tissue contact measurements as a feedback signal to impose a software-configured selective compliance at the device tip as the user advances the device. For example, the device tip may be programmed to become more or less compliant (e.g. automatically varying tension in pull wires used to articulate the device in bending, automatically activating stiffening mechanisms, etc.) based on detected tissue contact. In some examples, such selective compliance may depend on time, phase of the procedure, inputs from the user, and other variables.

The one or more navigation sensors 1006 may further include one or more localization sensors to provide position and/or orientation data describing the distal end portion 1018 of the elongate device 1002 including shape sensors, which may be substantially similar to shape sensor 108 described above, such as one of various fiber optic bend sensors (e.g., Fiber Bragg Grating sensors) and/or one or more antennas, electrodes, and electromagnetic sensors. Shape sensor data may be used to determine unexpected changes in device shape. For example, based on device kinematics and commanded device position (e.g. actuated pull wire tension), a calculated, expected shape of the device may be compared with a measured shape to determine that the device is making contact with tissue. Based on a pre-determined threshold differential between expected and measured shape, the device may be automatically repositioned or automatically made less compliant until the differential is reduced to an acceptable amount.

The one or more navigation sensors 1006 may optionally further include acid/base (pH) sensors and/or pressure sensors to aid with navigation. Such sensors may be used to sense transitions from one portion of the digestive system to the next, which are associated with pH and pressure changes. For example, in an embodiment, an array of pH sensors is located at or near the distal end portion 1018 of the elongate device 1002. The array may be arranged substantially perpendicular to a flow direction in the GI tract. The array measures the pH gradient as the device moves within the different regions of the GI tract, with higher pH density gradients being correlated to a desired flow direction, and the pose of the distal end portion 1018 may be manually or automatically adjusted based on the desired flow direction. While in the stomach, the pH sensors may detect the pH gradient in the direction of the pylorus sphincter (e.g., the pH gradient flowing toward and through the pylorus sphincter). Also while in the stomach, the pressure sensors may detect a pressure gradient in the direction of the pylorus sphincter (e.g., the pressure gradient flowing toward and through the pylorus sphincter) or in the direction of the transition between duodenum and jejunum at the ligament of treitz. The one or more navigation sensors 1006 may optionally further include temperature sensors to detect flow based on different temperatures in various regions of the GI tract (e.g., in the stomach versus the intestines).

As further shown in FIGS. 10-12A, the one or more navigation sensors 1006 may also include one or more gravity field sensors, such as inertial measurement units (IMU) or accelerometers or inclinometers to help properly align the distal tip of the elongate device 1002 in order to traverse the pylorus. At least one gravity field sensors are provided at the elongate device 1002 (e.g., at the distal end portion 1018 of the device 1002). In some examples, a patient reference gravity field sensor may be rigidly attached to an external portion of the patient body near a bony prominence (e.g. sternum) that has minimal relative movement with respect to the pylorus during patient positioning. The patient reference gravity field sensor provides a patient body frame of reference for alignment that indicates the pose of the patient. Based on the placement of the patient reference gravity field sensor and an estimated alignment of the pylorus with respect to the said bony prominence (for example, using bone-embedded anatomical reference frames), a target alignment of the gravity field sensor at the distal end of the device with respect to the reference sensor is determined. The control system can then determine a phase of the procedure when the device needs to be aligned to the pylorus or ligament of treitz (based on events) and activate an orientation control mode where the device tip servos to minimize the error between its attitude and the said target alignment. In some examples when only a gravity direction is available, the attitude may be defined as a direction vector with roll about the pointing direction specified arbitrarily. In yet other examples when a full coordinate frame is available such as in the case of an IMU, attitude may be defined as a set of three Euler angles or quaternion or other equivalent representations. In some examples, a plurality of gravity field sensors may be attached to the patient externally and along the device on multiple segments. In some examples, when the pylorus orientation with respect to gravity is relatively well-known based on patient positioning (e.g. lateral decubitus), a reference sensor may not be necessary. In some examples, the orientation controller may be supplemented by a position controller acting in response to another sensor (e.g. vision) to provide translational commands to approach the pylorus. In some examples, data from one or more gravity field sensors located along the elongate device may be used in the control system by using the data to calculate an instantaneous position and pose for each gravity field sensor along the elongate device. That series of positional and pose information may be used by the control system to enable the positioning of the device relative to the anatomy. The data may be used to supplement data obtained from images such as SLAM type camera pose information.

In some embodiments, navigation through the pylorus is further enhanced by using image analyses. The imaging sensors 1030 in conjunction with the visualization system 131 and display system 110 are used to track alignment of the elongate device 1002 relative to the pylorus and or ligament of treitz and provide navigational guidance. Feedback such as visual, audible, and/or vibration may be provided to the user for navigational guidance. For example, visible arrows or other directional visual analog information may be overlaid on a real time surgical view (e.g., endoscopic view) provided by the imaging sensors 1030 to identify misalignment between the anticipated axis of advancement of distal end portion 1018 and the desired axis of advancement through the pylorus and/or ligament of treitz. The directional visual information can indicate proposed corrective action to a user to correct the misalignment between the distal end portion 1018 and the pylorus and/or ligament of treitz and thereby help enhance navigational alignment (e.g., by maintaining centerline alignment to prevent or minimize undesirable contact of the elongate device 1002 with the lumen sidewall distal of the pylorus).

In some embodiments, fluid detection in real-time images may be used to assist with the navigation. For example, gravity will settle liquid at the bottom portion of the stomach, and this information may be used to help orient the user's navigation toward the pylorus. Image analysis identifies the settled liquid and so determines a gravitational field direction relative to the distal end portion 1018 and the associated endoscopic image.

In some embodiments, an algorithm relying on a neural network model may be trained based on a plurality of images of the pylorus or transition from the duodenum to jejunum at the ligament of treitz. Such an algorithm may help in identifying the opening of the pylorus or transition of the duodenum to jejunum in order to easily traverse the device toward the opening and past the point of suspension at the ligament of treitz. In some examples, the device may approach the pylorus opening along the lesser curve of the stomach in order to reduce its path length. In some embodiments, the neural network classifier may be trained to segment other anatomical landmarks relevant to the procedure administered by the device. These landmarks may include upper esophagus sphincter, lower curve of the stomach, pyloric sphincter, duodenal bulb, pancreatic sphincter, duodenum, jejunum, ligament of treitz, plicae circulares and other similar landmarks. In some examples, in addition to applying any suitable black box machine learning classifier to detect or assist in detecting the pylorus, traditional image processing techniques such as contrast enhancement and edge detection may be used for special cases such as the need to identify double vs. single pylorus. In some examples, a single pylorus may be detected as a continuous closed area in the processed image and a double pylorus may be detected as partitioned areas. In some examples, the temporal and spatial rate of change of detected areas consistent with the pylorus may be used by the control system to determine the ideal location and time of entry into the pylorus.

In some embodiments, an audio field measurement is used to detect the pyloric channel and ligament of treitz by detecting unique or characteristic sounds or sound gradients. The audio field measurement may be provided by an intra-luminal microphone mounted, for example, at or near distal end portion 1018. Such a detector may be used, like the previous sensors, to detect other lumen restrictions, such as the pancreatic sphincter, plicae circulares or ileocecal valve. In some examples, the audio-field measurement may provide cues for desired motions of the device based on a direction of flow of fluids, opening and closing of sphincters, and peristaltic motions, among other relevant aspects of the anatomy of interest.

### Aligning and Navigating Through the Duodenum and the Jejunum

When a manually controlled treatment device is navigated through the duodenum, there is a danger of damaging or perforating the duodenum wall as the device moves through the proximal bulb of the duodenum, generally identified as the first three regions (D1, D2, and D3 in FIG. 9) of the duodenum's four regions. This portion of the duodenum has a difficult C-shaped path to navigate, sometimes compounded by tortuosity in a sub-segment of patients. The upper and lower duodenum regions are problematic areas to navigate while obtaining proper angles with an endoscope in order to visualize both the mucosal surface in the duodenum for possible treatment and critical anatomy. Navigating through the duodenum is also difficult because applying an excessive amount of force to the duodenum wall can damage the duodenum surrounding areas and structures (e.g., the ligament of Treitz). In addition, navigation into the distal portion of the duodenum (e.g., D3 and D4) becomes more challenging because the required treatment path length from the mouth to the jejunum is approximately 100-150 cm with longer path lengths needed for tools working via channels in the instrument requiring path lengths of 200-260 cm. Therefore, although it is desirable and advantageous to reduce this path length by keeping the body of the device closer to the lesser curve of the stomach, staying close to the lesser curve is difficult because the stomach typically has a sharp curve near the lesser bend, in contrast with a more open, gentle curve near the greater bend.

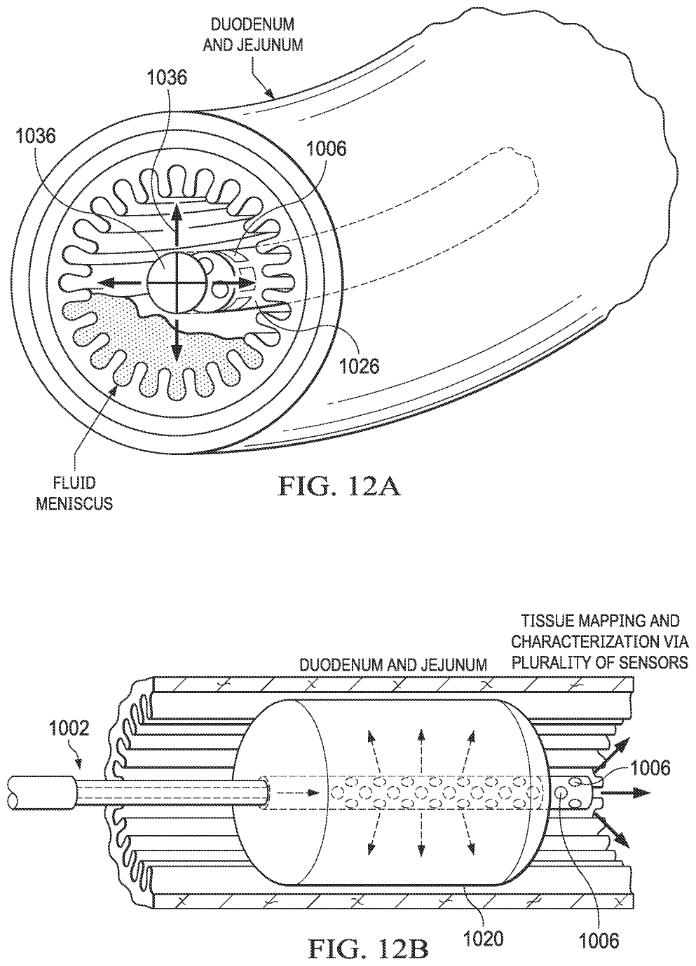

With reference to FIGS. 12A-12B, aspects of the present disclosure provide navigation and control of a computer-assisted teleoperated treatment device through portions or the entirety of the duodenum, including regions D1-D4, and optionally through at least the proximal portion of the jejunum. As described herein, the teleoperated system is able to re-center the elongate device 1002 within the center of the duodenum lumen and/or the jejunum lumen to prevent or minimize contact with the lumen walls and so reduce inadvertent trauma, such as perforation, while reducing the path length along the lesser curve of the stomach. Further, the teleoperated system is able to reduce the path length required for the treatment device to reach the target areas by staying closer to the lesser curve of the stomach. Additionally or alternatively, portions of the treatment device may be stiffened or rigidized to minimize drag or contact with the wall of the lumen. Such rigidization may be independent of the centering or may be used in conjunction with the centering.

In some embodiments, the navigation sensors 1006 and the imaging sensors 1030 described above are used to detect whether the elongate device 1002 is near or contacts the lumen sidewalls. In some embodiments, sensing may be based on detecting tissue contact by sensing (e.g., measuring) strain or friction between the elongate device 1002 and the sidewalls of the stomach, duodenum, and/or jejunum. Such strain sensing may include the strain gauges or other force transducer described above. In some embodiments, the navigation sensors 1006 and the imaging sensors 1030 may detect proximity to the lumen sidewalls by using an imaging-based approach. For example, the imaging sensors 1030 may be used to sense (e.g., detect or measure) a position or orientation of the device 1002 within the lumen. Optionally, in some embodiments, the imaging sensors 1030 may sense a misalignment between a desired orientation of advancement and the device 1002 (such as, for example, an approximate center of the lumen). In various embodiments, the navigation sensors 1006 and the imaging sensors 1030 may include one or more of an ultrasound probe (e.g., a radial ultrasound probe), a vision probe (e.g., via a camera or endoscope), a laser probe, an infrared probe, OCT sensors. GPS-like data using localization and/or shape sensors, or other distance sensor (e.g., a time-of-flight sensor). In some embodiments, a gravity sensor (e.g., sensor 1006) as described above may be used while in the lumen to help orient the surgeon or a computer control system for the device (see FIG. 12A). For example, an IMU or accelerometer on the elongate device 1002 may be used in combination with one or more external sensors (e.g., IMUs/accelerometers) on the patient and/or examination table to confirm the pose of the device or the distal end of the device. A reference IMU/inclinometer may be provided at the entry point into the body or may be otherwise attached to the patient. As another example, visual sensing as described above may be provided based on detecting a fluid meniscus in the lumen relative to the positioning of the patient (most often the lateral decubitus position for an upper endoscopy) in combination with the one or more external sensors on the patient and/or examination table. In some embodiments, the various sensors described above may be used individually or in combination. In some embodiments, one or more of visual, audible, and/or vibration feedback may be provided to the user to identify proximity to, or contact with, the lumen wall, and/or may detect misalignment relative to a desired orientation of advancement within the lumen (such as an approximate center of the lumen). To assist with the orientation, indicators 1036 such as vectors, arrows, crosshairs, or other visual orientation markers may be overlaid or incorporated into a graphical user interface to help center the elongated device with in the lumen or otherwise guide the advancement and orientation of the device. In some embodiments, proximity or contact to the lumen sidewalls and/or misalignment with a desired orientation of advancement may be detected by one or more of an ultrasound probe (e.g., a radial ultrasound probe), a vision probe (e.g., via a camera or endoscope), a laser probe, an infrared probe. GPS-like data, or other distance sensor (e.g., a time-of-flight sensor). For example, such sensors may detect the position of the device 1002 based on relative distances between the device 1002 and the walls of the lumen. For example, the sensors may detect that a portion of the device 1002 is closer to a first portion of the lumen walls and is farther from other portions of the lumen walls, which may exceed a predetermined threshold or ratio of misalignment based on a diameter of a section of detected lumen.

The navigation sensors 1006 and the imaging sensors 1030 may be used to manually or automatically re-center the elongate device 1002 within the lumens, for example, based on detected proximity or contact with the lumen walls and/or based on misalignment with a desired orientation of advancement (such as an approximate center of the lumen). Such re-centering optionally may be applied to the distal end portion 1018, one or more sections of the elongate device 1002 that are proximal to the distal end portion 1018, or both the distal end portion 1018 and one or more proximal sections.

In some embodiments, the clinical operator may apply manual re-centering based on visual, audible, and/or vibration feedback indicating proximity of, or contact with, one or more portions of the elongate device 1002 to the lumen wall and/or based on misalignment with a desired orientation of advancement. For example, feedback may be provided to the user via haptic feedback to the user control, or visual feedback may be provided to the display system 110 (e.g., via a graphical user interface). Such feedback may indicate, for example, increased strain on the device and the need to re-center the device within the lumen. Such feedback may include, for example, visual information that advancement is near the walls of the lumen or that advancement is moving closer to the walls of the lumen. Such feedback may include, for example, a misalignment between the device 1002 and the walls of the lumen. The user can then manually re-center the elongate device 1002 within the lumen based on the feedback. Likewise, the clinical user may use the provided feedback to center the device along any defined desired line of advancement or withdrawal. In some examples, if an anatomic model is available (e.g., based on upper GI endoscopy followed with barium to map the anatomy, based on CT imaging, or based on other survey or endoscopic data) correction of the device 1002 may utilize the model. In some examples, a target GUI may be provided showing a target icon overlaid on a forward-looking view of the lumen, and the current position of the center of the device 1002 may be determined using any of the sensors described above. The target icon (e.g., cross hairs) could be positioned based on an optimal driving path (e.g., if path was around a bend, then the cross hairs would be shown closer to a side wall). If the path is near an area of sensitive anatomy, the cross hairs may be centered.

In some embodiments, automated or autonomous re-centering may be applied based on detected proximity or contact to the lumen sidewalls or based on misalignment with a desired orientation of advancement (e.g., relative to an approximate center of the lumen) by one or more of an ultrasound probe (e.g., a radial ultrasound probe), a vision probe (e.g., via a camera or endoscope), a laser probe, an infrared probe. GPS-like data, or other distance sensor (e.g., a time-of-flight sensor).

In some embodiments, manual or automated re-centering may be applied independent of detection of proximity or contact to the lumen sidewalls. For example, re-centering may occur manually or automatically based on the time or phase of the procedure, or insertion amount of the elongate device 1002. For example, manual or automatic re-centering may occur when the distal end portion 1018 of the elongate device 1002 has reached certain intervals, such as entering regions D1, D2, D3, or D4 of the duodenum or the proximal jejunum at the ligament of treitz. In some embodiments, manual or automatic re-center may occur at regular or irregular intervals.

With reference to FIG. 12B, in some example embodiments, a re-centering mechanism is based on one or more expandable members 1020 that expand radially outward from the elongate device 1002 to contact the lumen sidewall. The re-centering mechanism may allow the device to be re-centered and then navigation may be continued through the lumen following re-centering. The expandable member 1020 may have an approximately uniform radial expansion such that the elongate device 1002 will become centered (e.g., re-centered) within the expandable member 1020. The expandable member 1020 may be located near the distal end portion 1018 or proximal to the distal end portion 1018. Further, a plurality of two, three, or more expandable members 1020 may be provided along the length of the elongate device 1002. The expandable member 1020 may be used for proximal portion, middle portion, and/or distal portion centering of the elongate device 1002 within a body lumen. In some embodiments, the expandable member 1020 may be located along the length of the flexible body 1016 and/or may be fixed to the instruments 1026. In some embodiments, the expandable member 1020 may be an inflatable balloon (e.g., gas and/or liquid (e.g., saline) filled) that expands in the lumen or in the device itself. In some other example embodiments, the expandable member 1020 may include an expandable mesh member or expandable stent member. The expandable member 1020 may also serve to provide therapeutic treatment to target regions, as described in further detail below.

In some embodiments, a re-centering mechanism may be based on cable, linkage, or other steering control mechanism of portions of the flexible body 1016. For example, the flexible body 1016 may include a plurality of control cables or linkages embedded in a wall of the flexible body 1016. The plurality of control cables or linkages run circumferentially along the length of the flexible body and are selectively controlled by tension and release to adjust the pose of the flexible body 1016 within the duodenum lumen.

In some embodiments, as the elongate device 1002 is advanced or withdrawn in the duodenum or jejunum, friction between the elongate device 1002 and the intestinal sidewall is minimized to reduce the potential for tissue damage as the device is advanced or withdrawn. In some embodiments, dithering motions (e.g., forward/backward movement and/or vibration movement) may be applied to reduce trauma. In one aspect such dithering includes an inchworm-like motion in which the device is advanced/withdrawn in a desired direction for a first distance, then withdrawn/advanced in an opposite direction for a second distance smaller than the first distance, and then once again advanced/withdrawn in the desired direction for the first distance. In some embodiments, other small amplitude mechanical vibrations may be applied to reduce friction, such as axial vibrations along the length of the flexible body 1016, radial vibrations, or a combination of axial and radial vibrations.

In some embodiments, the elongate device 1002 may be selectively stiffened or rigidized along one or more curvatures of the body (e.g., the stomach, the duodenum, and/or the jejunum) to maintain desired centering at individual locations in space within the body. Portions of the device 1002 may be stiffened or rigidized to minimize drag or contact with the wall of the lumen and/or to maintain relative positioning or alignment within the lumen. Such stiffening or rigidization may optionally be used in conjunction with the re-centering mechanisms described herein. For example, a re-centering mechanism may be used to center the device 1002 within the lumen. After centering the device 1002 within the lumen, portions of the device 1002 (e.g., a proximal portion and/or a distal portion) may be selectively stiffened or rigidized. A sequence of re-centering following by selective stiffening or rigidization may be manually or automatically engaged, for example, by a user selecting an operation for the sequence via an input device and the sequence occurs under computer control (such as via control system 812). The user may select one or more locations for re-centering (e.g., based on available locations of the expandable members 1020), or the locations may be automatically selected based on detected proximity or contact to the lumen sidewalls or based on misalignment with a desired orientation of advancement. The user may additionally select whether to rigidize or stiffen the device after re-centering, and the user may select locations for rigidization. Additionally or alternatively, the control system may apply a rigidizing system including automatically applying selective rigidization following re-centering (e.g., by selectively rigidizing at or near the locations of the expandable members 1020 or any other portions). In some embodiments, the user may select re-centering and the control system automatically deploys re-centering and automatically follows the re-centering deployment with selective stiffening or rigidization. In some embodiments, the elongate device 1002 may be selectively stiffened when the distal end is in the stomach or in the duodenum to aid with navigation. Examples of stiffening mechanisms are provided in PCT Patent Application PCT/US/2019/037954, filed Jun. 19, 2019 (disclosing “Systems and Methods for Holding a Flexible Elongate Device in a Pose”), which is incorporated by reference herein in its entirety. For example, one or more stiffening mechanisms may include expandable members that expand to engage a support structure in the flexible body 1016 to maintain the support structure in a desired pose. For example, one or more stiffening mechanisms may include a plurality of control wires or cables (e.g., optionally nitinol wires) embedded in a wall of the flexible body 1016, as described above. The plurality of wires may be selectively engaged or articulated to maintain a pose. In some embodiments, the one or more curves in the device are propagated along the length of the device as the device is advanced or withdrawn. And, one or more stiffened portions may be similarly propagated along the length of the device as the device is advanced or withdrawn. In some embodiments, the mechanism may be combined with a wound fiber optic fiber to provide strain stiffening feedback. As shown in FIG. 12B and as described in more detail below, optionally, the curvatures of the body spaces may be mapped to characterize the curvatures for navigation and to identify target areas for treatment. The tissue mapping and characterization may be performed using information received, for example, from the sensors 1006. Selective control of the elongate device 1002 may then be provided based on the device 1002 insertion depth corresponding to the curvatures of the body spaces.

In some examples, positioning the elongate device 1002 closer to one wall than another may be preferable to centering the device 1002. A path of travel may be determined to minimize length and/or contact forces between the elongate device 1002 and the wall of the anatomic lumen. For example, driving the elongate device 1002 closer to the inner path may reduce the overall path length to travel. A determination may be made as to where the device is within the lumen and then position the elongate device close to the wall within a threshold distance to, for example, provide an offset to avoid tissue damage. In some examples, the shape of the device may be determined to detect a corresponding bend in anatomy, and then the path of travel may be adjusted to be closer to the inner bend. Any of a variety of proximity, force, imaging, or other sensing system may be used to ensure that the elongate device is not too close to the wall.

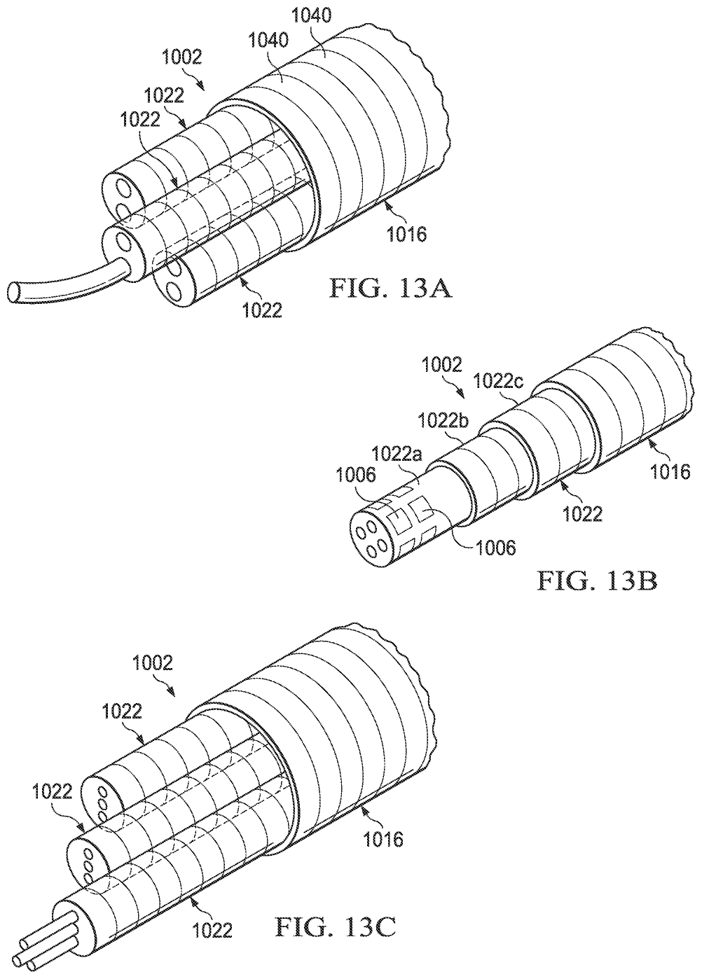

With reference to FIG. 13A-13C, the elongate device 1002 may include multiple bending sections 1040 to provide selective stiffening. In some embodiments, as shown in FIGS. 13A and 13C, the flexible body 1016 may include a plurality of abutting articulating sections (e.g., two sections, three sections, or more) with preconfigured (e.g., predefined) shapes to traverse the C-shaped path of the duodenum. The abutting articulating sections may have different bend radii from each other. In some embodiments, as shown in FIG. 13B, the elongate device 1002 may include a plurality of telescoping catheters 1022, 1022a, 1022b, 1022c (e.g., guide tubes) (e.g., two, three, or more catheters). The telescoping catheters 1022 may be arranged concentrically. In one example, an inner catheter 1022a having a relatively smaller diameter extends distally from the distal end of an outer catheter 1022b having a larger diameter. In another example, an innermost catheter 1022a having a relatively smaller diameter extends distally from the distal end of a middle catheter 1022b having a larger diameter, and from an outermost catheter 1022b having a larger diameter than catheters 1022a, 1022b. In some embodiments, the concentric catheters may include sections that may have different bend radii from each other and may be selectively advanced through the bends of the duodenum. The concentric sections may have different pre-defined stiffened shapes corresponding to the different bends of the duodenum, jejunum, and/or the lesser curve of the stomach. In other embodiments, each concentric catheter may have a different stiffness. For example, the outermost catheter 1022c may be constructed of stiffer materials than the inner catheters 1022a, 1022b such that outermost catheter 1022c may act as a stable delivery platform to reduce potential damage on surrounding tissue. In other embodiments, an inner catheter (e.g., 1022a and/or 1022b) may be stiffer than an outer catheter (e.g., 1022c). Optionally, preoperative imaging may be used to help identify the proper bend section shapes to use. For example, the concentric sections may have various pre-defined shapes based on a neural network assessment of prior endoscope measurements and the patients height with bends optimized at each anticipated transition in the anatomy from the mouth to the esophagus, fundus of the stomach, antrum of the stomach, pylorus, first portion of the duodenum, second portion of the duodenum, third portion of the duodenum, fourth portion of the duodenum, ligament of Treitz. and proximal portion of the jejunum. In another example, a model based on pre-operative imaging or on intra-operative, pre-procedural mapping may be used to identify lesser curves of the stomach either automatically or as defined by a user.

In some embodiments, the catheters 1022 may be used in conjunction with or more expandable members (such as expandable members 1020 described above) to allow for centering of the device 1002 in the lumen and advancement of the device 1002. In one example, the flexible body 1016 may have at least one catheter 1022 and at least one expandable member that may be expanded from the flexible body 1016 to contact the lumen wall. The expandable member may be expanded for centering the device 1002 in the lumen, and the catheter 1022 may be advanced while the expandable member holds the device 1002 centered within the lumen. After the catheter 1022 is advanced, the expandable member may be collapsed to remove contact from the wall, and the flexible body 1016 is advanced past the distal end of the catheter 1022. The catheter 1022 may have a stiff structure, or otherwise be selectively stiffened via one or more stiffening mechanisms as described herein, to provide rigidity to the device 1002 such that the device 1002 may remain substantially centered in the lumen when the expandable member is compressed and the flexible body 1016 is advanced. After the flexible body 1016 is advanced to a new position, the process may repeat. In some embodiments, this advancing process may occur automatically by a user selecting an operation for an advancing sequence via an input device and the sequence occurs under computer control (such as via control system 812). Optionally, in some embodiments, a user may select a manual or automated sequence of advancing, for example via a “move forward” command at the master assembly 806. Once selected, the advancement process may proceed by a series of steps under computer control including one or more of: centering the device 1002 in the lumen, automatic stiffening, and advancement of the device 1002, and repeating the process. In some embodiments, the advancement process may proceed through some portions of the lumen by centering the device 1002 followed by advancement of the device 1002 without automatic stiffening. In some embodiments, the advancement process may proceed through some portions of the lumen by stiffening of the device followed by advancement of the device 1002 without centering the device 1002. In some embodiments, the advancement process may proceed through some portions of the lumen by centering the device, stiffening of the device followed by advancement of the device 1002. In some embodiments, centering the device and/or stiffening the device may occur at regular intervals and/or may be based on a detected proximity or contact to the lumen sidewalls and/or based on misalignment with a desired orientation of advancement. In another example, the flexible body 1016 may have two or more concentric catheters, such as the inner catheter 1022a with a relatively smaller diameter and the outer catheter 1022b with a larger diameter. The outer catheter 1022b may have the expandable member and the inner catheter 1022 may have the stiff structure or be selectively stiffener.

With further reference to FIGS. 13A and 13C, in some embodiments multiple catheters 1022 may be arranged side-by-side within the flexible body 1016 (e.g., two, three, or more catheters). The multiple catheters 1022 may include a primary or lead catheter and a plurality of trailing catheters. The primary catheter may have a loop to selectively engage and drive the trailing catheters, which follow the lead catheter. The lead catheter may be small and nimble, with a large amount of flexibility. The plurality of trailing catheters may be stiffer than the lead catheter and may be selectively engaged with the lead catheter (e.g., to provide stiffness to the lead catheter). In some embodiments, the lead catheter has controlled articulation, and the trailing catheters are passive. The trailing catheters may carry end effector mechanisms to perform various tasks within the lumen, including navigation, sensing, tissue characterization, mapping of the anatomy, energy delivery, and confirmation of tissue change post-therapy. The trailing catheters may be optimized for rapid exchange (i.e., exchanging a first trailing catheter with a second trailing catheter) and for connection to the lead catheter. In some embodiments, all of the controlled articulation is provided in the lead flexible catheter, and the trailing stiffer catheters provide rigidity. It should be noted that aspects of rigidity are not necessarily unbendable, and instead rigid devices may be sufficiently stiff to provide sufficient rigidity to perform a clinical task. In some aspects, a small degree of flexibility may be desired to provide some elasticity within the soft tissue environment. The trailing catheters may follow the lead catheter and be activated (e.g., selectively engaged with the lead catheter) for optimal path length based on geometry within the hollow viscous structures and on sensor feedback. In an alternative arrangement, the lead catheter may be steered using a guidewire containing shape sensing or electromagnetic shaping elements to aid in steering and guiding the guidewire inserted in a lumen of or integrated within the lead catheter. The multiple catheters 1022 and the flexible body 1016 may include one or more sensing elements, such as navigation sensors 1006, to aid with navigation. Such sensing elements may be provided on each of the catheters 1022, and may include, for example, pH (acid/base), pressure. OCT, ultrasound, fiber optic, strain gauge, etc. sensors. Such sensors may be provided to enable proximal portion, middle portion, and distal portion or end centering of the catheters 1022 within a body lumen. For example, OCT sensors may be provided on a distal end or sides of the catheters 1022 for mapping, capacitive proximity sensing may be provided more proximally on the catheters 1022 to determine how close the catheters 1022 are to the lumen walls, and/or shape or strain sensing sensors may be provided even more proximally on the catheters 1022 to measure forces against the lumen walls. Re-centering may then be applied as described above. For example, proximal capacitive sensing and orientation and alignment cues from an inclinometer may be used to help automatically conform to one or more of the desired curves of the G1 tract.

Accounting for Motion of Patient Anatomy when Providing Treatment

When a treatment device is navigated through the digestive system, movement of the patient's diaphragm, distension of hollow viscous structures as well as peristaltic waves through the GI tract can make it difficult to provide the precise treatment needed. Peristalsis is the involuntary movement of the longitudinal and circular muscles located within the walls of the esophagus, stomach, and intestine (activated by motor neurons located within the gastrointestinal wall, otherwise known as the enteric or intrinsic nervous system (ENS)). Peristalsis, driven by the intestine's local ENS, results in waves which propagate passage of contents forward in order to aid mechanical digestion while optimizing contact with bile acids and pancreatic enzymes secreted from the ampulla of Vater and thus nutrient contact with the mucosal lining of the intestines. Peristalsis is problematic when performing endoscopy or placement of devices because it can result in migration of the treatment instrument, which in turn affects the precision of the instrument placement in the treatment zone. If peristalsis disrupts a treatment in progress, critical structures could be inadvertently injured, including the esophagus, bulb of the duodenum, the ampulla of Vater, the intestinal wall, or the segment of the intestine anchored by the ligament of Treitz.

Peristaltic waves are triggered by the local intrinsic nerves of the intestine (motor neurons located within the enteric plexus of the gastrointestinal wall). Sympathetic output causes waves to slow in frequency, whereas parasympathetic output causes the waves to increase in frequency. It is therefore beneficial to attempt to synchronize treatment with these waves in order to improve treatment application and accuracy.

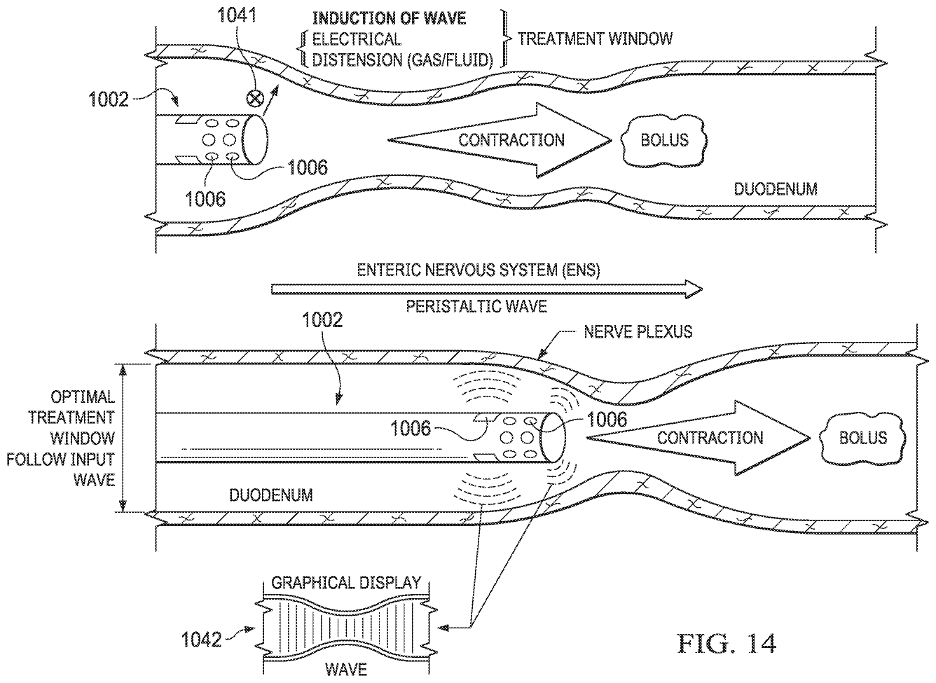

Referring to FIG. 14, in some embodiments, peristaltic waves are anticipated by using sensors 1006 on the elongate device 1002 to sense wave patterns and then automatically delay treatment or otherwise predict treatment windows. Such peristaltic wave sensors may include one or more of stretch sensors, strain gauge transducers, manometers (manometry), accelerometers, or enteric nerve monitoring by monitoring membrane potentials with multisite optical recording techniques (e.g., voltage sensor, voltage sensitive dyes) or electromyographic monitors. The monitored waves may be displayed on a graphical display 1042. Peristaltic wave monitoring may also be provided via the changes in pressure (gradients) by using a plurality of pressure (e.g., absolute, gauge differential) sensors (e.g., piezoelectric, optical. MEMS, resistive, capacitive) placed at the distal end and/or throughout the outer body of the elongate device 1002. In addition, pH (acid/base) sensors may be used to measure the amount of bile acids and pancreatic enzymes secreted into the intestine during peristalsis. In some embodiments, anchored expandable elastic elements (e.g., balloons) with embedded stretch, force or accelerometer sensors may be used to sense peristaltic waves and thereby estimate peristaltic wave patterns. In some embodiments, based on the sensing described herein, treatment or advancement may be halted during the period of a peristaltic wave. In some embodiments, time windows for treatment and/or advancement of the device may be estimated based on estimating peristaltic wave patterns using the sensing described herein (e.g., based on sensing multiple waves and estimating time windows between waves). Safe time margins are identified within the windows, and treatment is subsequently applied within the time margin boundaries. Optionally, in some embodiments, a deep neural network (DNN) model is trained on peristaltic motion prediction based on sensor inputs to more accurately identify windows of treatment. For example, the DNN receives sensor input, uses the sensor input to predict a quiet time window of a first-time duration before the next peristaltic wave, and then defines a statistically safe treatment margin time window of a second time duration within the first time duration. Treatment is then applied (either automatically or an indicator is provided to the user to initiate ablation energy delivery) during the safe treatment margin time window with statistically good assurance that it will not be interrupted by the next peristaltic wave. In some embodiments, the treatment is delivered as a single event. In alternative embodiments, the treatment can be delivered in an inverse cyclical wave countering the cyclical peristaltic wave so that ablative energy is delivered with a varying amplitude where the highest energy delivery is provided within a center of the safe treatment window. The DNN constantly receives sensor input and modifies time window predictions accordingly in order to account for individual patient variations, peristalsis induced by environmental changes, etc.

In some embodiments, electrical stimulation 1041 from the elongate device 1002 may be used to induce a peristaltic wave with contraction of the smooth muscle in the intestine, thereby providing a treatment time window immediately after the induced wave. The time window is based on statistically safe prediction. Use of gas (e.g., air or carbon dioxide) in the bowel may also be used to trigger a peristalsis wave via luminal distension or induced smooth muscle contraction, which subsequently provides a time window of approximately 15 seconds to perform treatment. The treatment can be automatically applied during the time window for the duration of the time window or for a portion of the time window limiting overlap with peristaltic motion. For example, for a 15 second window, after 3 seconds have elapsed, treatment can be provided for a 9 second window, providing for a 3 second buffer at a beginning and end of the 15 second treatment window. In some embodiments, treatment may be scaled so that it increases from zero during a first portion of the treatment window, hits a maximum at a center of the treatment window, and decreases to zero towards the during a second portion of the treatment window.

In some embodiments the beginning of peristalsis is sensed, and then the system automatically suspends treatment. For example, one or more sensors located proximally on a device sense the beginning of peristalsis proximal of the treatment site, and treatment is then suspended at the distal end of the device until a safe treatment time window begins after the peristaltic wave passes the treatment function at the distal end of the device. In some embodiments, shape sensor data can be used to determine peristalsis along the device by determining tissue contact along the device as it is navigated through anatomy. The measured peristaltic wave can be used to predict when movement will be experienced at an ablation target site to establish the treatment window without anatomical movement at the treatment site.

In some embodiments, stimulation/excitation of the nerve plexus within walls of the duodenum is used to reach a threshold to pause peristaltic activity. Nerve stimulating sensors (e.g., electrodes) may be included along the length of the elongate device 1002, included in catheters 1022, or delivered via a separate device.

Providing Real Time Tissue Characterization and Mapping of Target Regions for Treatment, and Selectively Providing Treatment within the Target Regions while Protecting Sensitive Regions; Providing Treatment to a Longer Treatment Path

When providing a treatment device to the stomach, duodenum, and/or jejunum, various treatments can provide selectively targeting certain structures while protecting adjacent structures. One treatment includes targeting the mucosal layer for treatment while protecting the outer layers of the gut (i.e., the submucosal or musculature (strength) layer) via a lifting procedure in which a fluid (e.g., saline) or gas (e.g., carbon dioxide) and/or a combination of fluid and gas is injected in the submucosal layer order to raise the inner mucosal layer away from the submucosal layer, thus preventing damage to the adjacent outer submucosal layer of the organ. Another treatment includes selectively targeting the underlying enteric nerve plexus for treatment (e.g., removal of portions of the nerve plexus in the submucosal layer). Another constraint is needed to avoid applying treatment to the ampulla of Vater or any aberrant minor ducts opening into the duodenum so that the hepatopancreatic sphincter (sphincter of Oddi) remains functional, and the patient does not suffer iatrogenic injury from pancreatitis, duodenal hemorrhage, dilation of the bile ducts/bile duct leakage, liver damage, spleen damage, or pancreas damage.

Further, some patients will have a tortuous or corkscrew shape of the duodenum and not a clear C-shape. This aberrant anatomy prevents the manual endoscopist from successfully navigating the initial portions of the duodenum and completing an evaluation and therapy within the proximal intestine because this anatomy is not obvious to the user prior to the procedure. Further, pre-operative imaging, such as upper-GI follow through studies. CT scans, or abdominal ultrasounds, are typically not routinely performed prior to these types of procedures, and the extra tortuosity of the corkscrew shape makes manual navigation through the duodenum extremely difficult.

In addition, manual techniques only target a limited amount of the mucosal layer in the distal fourth region of the duodenum past the duodenal papilla, which is traditionally marked with either a radiographic clip or by argon plasma energy source, and manual techniques often do not target the proximal regions of the duodenum. Nevertheless, it is known that treatment effectiveness is dose-dependent on the size of the treated area. By visualizing and/or mapping tissue areas to target, it is possible to access additional areas within the proximal intestine optimal for therapy (including one or more areas in the proximal duodenum, distal duodenum, areas past the ligament of Treitz, and/or areas in the proximal portion of the jejunum), which may provide in various embodiments, greater than 10 cms of treatment length, 10-20 cms of treatment length, or in some cases up to 20-30 cms of treatment length, and may significantly improve treatment effectiveness.

For treatment, as described above, it is optimal to remove or destroy (e.g., ablate) sub-millimeter to millimeter tissue depth ranges in the lumen wall in order to achieve adequate treatment, such as in some embodiments, by removal of a majority of the mucosal layer, and in further embodiments, possibly up to 75-95 percent of the mucosal layer. It also may be acceptable to treat into the submucosal layer, provided the underlying muscle layer is not reached and compromised.

In some embodiments, a multistage process is provided for treatment, involving an initial mapping step, a treatment step, and a post-treatment assessment. During the initial mapping step, pre-operative and intra-operative (real time) characterization of the duodenum and/or jejunum is provided to characterize and identify areas to treat, particularly areas of the mucosal layer containing a relatively higher density or amount of hypertrophic mucosa and/or enter-endocrine hyperplasia and/or nerve cells. During the treatment step, the identified hypertrophic and hyperplastic areas are treated as described further herein. During the post-treatment assessment step, which may optionally be performed in real-time during a procedure, immediately after a procedure, or days or weeks after a procedure, an assessment is performed to determine treatment effectiveness.

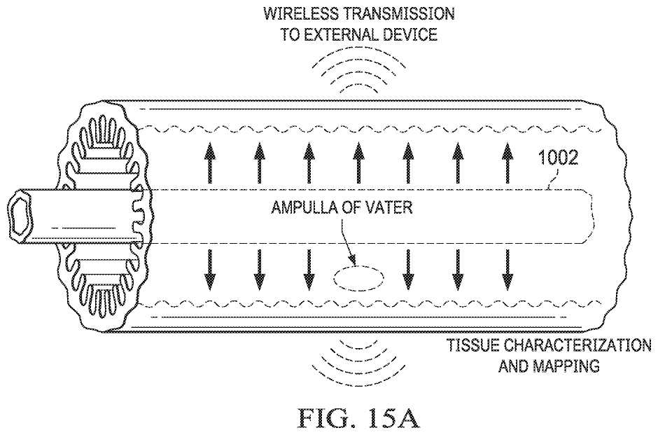

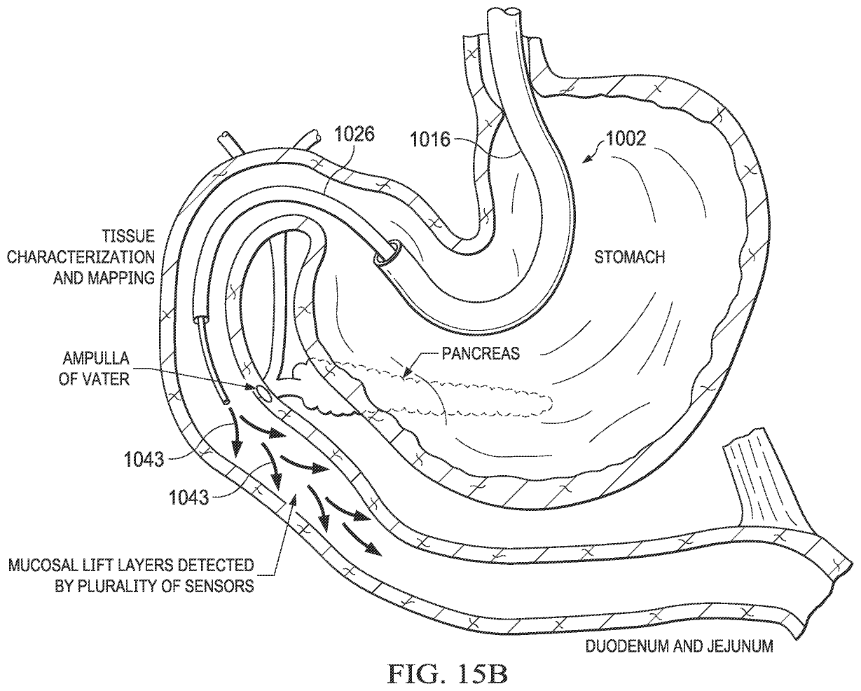

With reference to FIGS. 15A-B, for the pre-operative and intra-operative tissue characterization of areas for treatment, one or more tissue characterization sensors may be used to identify and characterize areas to treat as well as areas to avoid to prevent inadvertent injury. The tissue characterization sensors may include the navigation sensors 1006 and imaging sensors 1030 and other sensors as further described below. The tissue characterization sensors may include a single sensor or a plurality of sensors distributed along the length of the elongate device 1002 (e.g., along the length of the flexible body 1016). The tissue characterization sensors may be fixed to the instruments 1026 (e.g., catheters 1022). Optimal areas to treat hypertrophic mucosa and/or endocrine hyperplasia within the proximal intestine can then be determined with emphasis for treatment to the portion of the gut making contact with bile acids and pancreatic enzymes in the distal portion of the duodenum and proximal portion of the jejunum. Incretin hormones are gut peptides secreted after nutrient intake, and they stimulate insulin secretion together with hyperglycemia. Glucose-dependent insulinotropic polypeptide (GIP) and Glucagon-like peptide-1 (GLP-1) are the known incretin hormones from the upper (GIP (K cells)) and lower (GLP-1 (L cells)) gut.

An initial mapping may be performed to generate a 2D or 3D model of the desired surface areas of treatment by using the one or more sensors. Further data from the sensors is processed to create a 2D or 3D mosaic associated with the model and is plotted to help optimize the treatment zones. For example, optionally, planar images may be taken and used together to classify tissue as either an area to be treated or not treated. Optionally, software tagging (automatic or manual) of the inner folds of the intestinal lining (plicae circulares) during examination of the inner lining of the proximal intestine may be used in conjunction with a computer-generated segmented (manual or automatic) three-dimensional reconstruction of the intestine with or without external fiducials and sensors determining patient positioning. By using software tagging of internal anatomical landmarks in conjunction with surface area mapping of the duodenum (critical structures, surface area for treatment), an external view is created in conjunction with externally placed fiducial markers. This external view allows for an external overlay of the anatomy onto the patient and thus enhances the user's understanding and planning for treatment with relay via a graphical user interface on a screen or a heads-up display. After an initial depth map is generated, radial ultrasound or other sensors may be used at the time of treatment so that the depth map can be used to localize treatment.

In some embodiments, the tissue characterization sensing may sense peptide and hormone readings to identify areas of mucosal overgrowth, enteric nervous system activity and/or entero-endocrine hyerplasia resulting in down-regulation/inhibition and thus decreased amounts of incretin hormones. The sensors may include GLP-1 and GIP peptide sensors (e.g., fluorescent, electrochemical, protein, and peptide-based biosensors). In some embodiments, a plurality of such sensors may be provided to detect concentrations and thus mapping K and L cells within the mucosal lining or vasculature of the intestine. In some embodiments, fluorescent agents to adhere to incretin peptides within the intestinal vasculature may be administered preoperatively and are then visualized during the procedure with fluorescent endoscopy. In some embodiments, measurement of incretion hormones (GLP-1 and GIP) and enteric nerve activity within the lumen of the gut may be provided via a transient capsule ingested pre-procedure. A biodegradable micro-array of sensors may be provided within the biodegradable capsule (e.g., capsule 1032 of FIG. 11B) and may make contact with the intestinal wall. The sensors may then be injected directly into the vasculature of the intestinal wall via small needles optimized to enter the mucosa, submucosa and smooth muscle of the intestine. These leave-behind biodegradable sensors then wirelessly transmit signals to an external device 1034 (e.g., any suitable external receiver, such as a modified smartphone executing an appropriate application) to provide measurement of incretin, insulin, and glucose levels from the vasculature via a plurality of communication pathways (Bluetooth, infrared, etc.) over a period of time to aid with mapping of the areas to be targeted during treatment. In some embodiments these signals are detected with a receiver integrated within a shape sensing catheter, thus aiding with mapping in real-time to aid with targeted therapy. These signals optionally may be used in any combination with a number of sensors to aid with volumetric and three-dimensional mapping of the entero-endocrine or nerve cells within the intestine. The sensors optionally are re-injected post-procedure in order to track success or failure of the therapy. In some embodiments, the biodegradable ingestible capsule may be ingested post-procedure to detect incretin, insulin, and glucose levels for a period of time after treatment.

In some embodiments, the sensing may include a pre-operative map with CT and intra-operative mapping in situ with the computer-assisted telesurgical system (see FIG. 3).

In some embodiments, a capsule endoscope or capsule sensor 1032 (see FIG. 11B) is administered pre-procedure enabled with white light, NBI, fluorescent, and/or accelerometer sensors in order to map the topography of the proximal intestine and identify areas of hypertrophied mucosa, endocrine hyperplasia, enteric sub-mucosal nerves, and concentrations of L and K cells.

In some embodiments, narrow band imaging (NBI) (e.g., blue and green wavelengths of light) with the use of specific dyes may be used to identify particular cell types otherwise not apparent with gross visualization.

In some embodiments, sensing may further include molecular level imaging, confocal microscopic imaging, hyperspectral/spectral imaging, photoacoustic imaging for microscopic views, and spinning optical fiber tips. In some embodiments, nerves may be imaged by using light polarization or by using lasers to identify nerves in the submucosal plexus.

In some embodiments, the inner ridges of the plicae circulares may be used as physical encoders because they do not move much (roughly 0.5 mm for the ridges). For example, machine vision is used to identify individual plicae circulares locations as the imaging device advances or withdraws through the lumen, and the individual locations are then used as visible physical markers for subsequent navigation during advancement and withdrawal within the lumen. The individual locations are stored along with an anatomical model as described above. The clinical user may use the locations along with generated visible tags associated with the locations for navigation. Likewise, the computer-assisted system may again identify the locations by using machine vision as it navigates or assists the user to navigate.

In some embodiments, a neural network is used to identify hyperplastic abnormal mucosal cells within the proximal intestine based on supervised learning of historic data.

In some embodiments. OCT sensors may be used to identify particular cell types, including hyperplastic, hypertrophic and abnormal mucosal cells. In some embodiments, subsampled OCT methods are used to allow a user to visualize layers of anatomy and thus (i) differentiate cell layers by seeing past the superficial layer of the mucosa, and (ii) visualize the underlying layers of the intestine (1-3 mm in depth) to provide images of the tissue and optical densities via a graphical relay. In some aspects volumetric data contained in the same shape as a camera image is used to provide a three-dimensional output to the user showing such information. The subsampled OCT methods may probe at optical density of tissue and, by using polarization of the cells contained with the tissue layers, may in some conditions have the ability to distinguish and identify nerves from the surrounding tissue. In particular, the subsampled OCT methods may exploit gaps within the mucosal villi where different polarization characteristics exist, thus allowing identification of nerves and various cell types contained within each layer of the intestinal wall. Further, hypertrophied cell layer and areas of endocrine hyperplasia will vary in thickness along the length of the duodenum (from stomach to jejunal direction), such that knowing the cell layer depth adjacent the distal end of the device as the device moves through the organ helps determine correct treatment depth. In some embodiments, a scan may be performed helically or spiraled, and the resultant image is then digitally unwrapped to provide a 2D view of the entire scanned area. The 2D view can provide information to the operator regarding which areas to target or not target for treatment. Further, following treatment, a subsequent scan may be used to characterize effectiveness of the treatment. The purely optical method of sub-sampled OCT is advantageous because treatment (e.g., ablation-removal of tissue) and imaging can occur in real-time under direct visualization without OCT signal interference. In some embodiments, direct visualization of nerves may be provided with post-spectral processing to avoid the use of contrast dyes. In some embodiments, the sensors include one or more spinning OCT fiber tips contained within a balloon with a known distance on a shape sensing or electromagnetic catheter that is automatically or manually manipulated to visualize the walls of the intestine.

In some embodiments, the characterization sensors may include non-destructive mass spectrometry probes to sample in real-time and differentiate the cell types desired to be removed based on a classifier database developed to detect molecular footprints (unique charge-to-mass ratios) of hyperplastic over-active endocrine cells.

In some embodiments, the characterization sensors may include radial ultrasound at the tip or sides of the elongate device 1002 or on the instruments 1026, and it may be used to provide visual tomography of proximal intestine in relation to/identification of critical structures such as the ampulla of Vater, ligament of Treitz, accessory ducts, or comprised or at risk portions of the bowel (e.g., poor blood flow/anti-mesenteric surface of the bowel).

In some embodiments, the characterization sensors may include enzymatic sensors to detect bile acid or pancreatic enzyme presence from the ampulla of Vater (e.g., via a mass spectrometry probe).

In some embodiments, the characterization sensors may include pH sensors used to detect acid/base changes within the gastrointestinal tract, such as tracking pH gradient changes from the stomach through the duodenum.

In some embodiments, idocynanine green (ICG) is administered intravenously preoperatively or intra-operatively or directly injected into the local gastrointestinal tissue to determine the location(s) of the major (and minor if applicable) duodenal papilla (e.g., the ampulla of Vater) as a standalone mechanism or in conjunction with a machine learning system optimized to detect the papilla.

In some embodiments, endoscope insertion distance past a known body location, such as the pyloric sphincter, is used to support tissue characterization.

In some embodiments, a shape sensor is used as one of the characterization sensors. Optionally, shape sensor data for the entire circumference of a body lumen may be obtained in combination with depth sensors (e.g., OCT or ultrasound) to continually obtain information through the thickness of the tissue at different measurements along the length of the lumen. The depth sensors may be constantly rotating to obtain full circumferential information.

In some embodiments, stereoscopic vision, which may be obtained via optical sensors, is used as one of the characterization sensors. In some embodiments, the elongate device 1002 may include two side-by-side cameras, each capable of monoscopic and stereoscopic vision. The cameras may have for example a 3 mm diameter objective with an interpupillary distance of 3 mm or more to provide sufficient stereoscopic depth information. In some embodiments, a two-stage visual map of the duodenum is created by first passing the elongate device 1002 through the duodenum while performing stereoscopic imaging, followed by a second pass with monoscopic imaging to refine the map.

Bile and other fluids in the area can make it difficult for the operator to see and visualize treatment. Accordingly, in some embodiments, fluids are cleared during the initial mapping phase. For example, in some embodiments a camera with suction, or gas blowing, or both, is provided to clear liquids in order to get the best image possible and optionally construct the best vision-based mapping possible. In some embodiments, the elongate device 1002 performs a first pass through the treatment region to clear liquids. After the first pass, the elongate device is removed and reinserted under the improved vision. As desired, humidification is optionally provided to the gas used to clear liquid (or to otherwise insufflate the volume in front of the camera) to prevent tissue from drying, or alternately low-humidity gas is use to help dry or desiccate tissue. Tissue surface moisture can be sensed and subsequently adjusted depending on need under a desired treatment. In addition, certain imaging methods may be used to identify tissue surface moisture characteristics. In some embodiments, the elongate device 1002 may combine imaging, blowing, or suction automatically. For example, a sequence may be provided in which liquids are sprayed outward to irrigate a surface area, then cleared by blowing gas, and then followed by imaging the surface area and advancing the device. Once the area has been cleared and optionally imaged, a treatment device may be inserted and treatment provided.

Selectively Providing Treatment within the Target Regions while Protecting Sensitive Regions; Providing Treatment to a Longer Path Length

The present disclosure provides various treatments for selectively identifying and removing (e.g., ablating) target regions in the GI tract while protecting sensitive areas and while providing a longer path length of treatment for improved effectiveness.

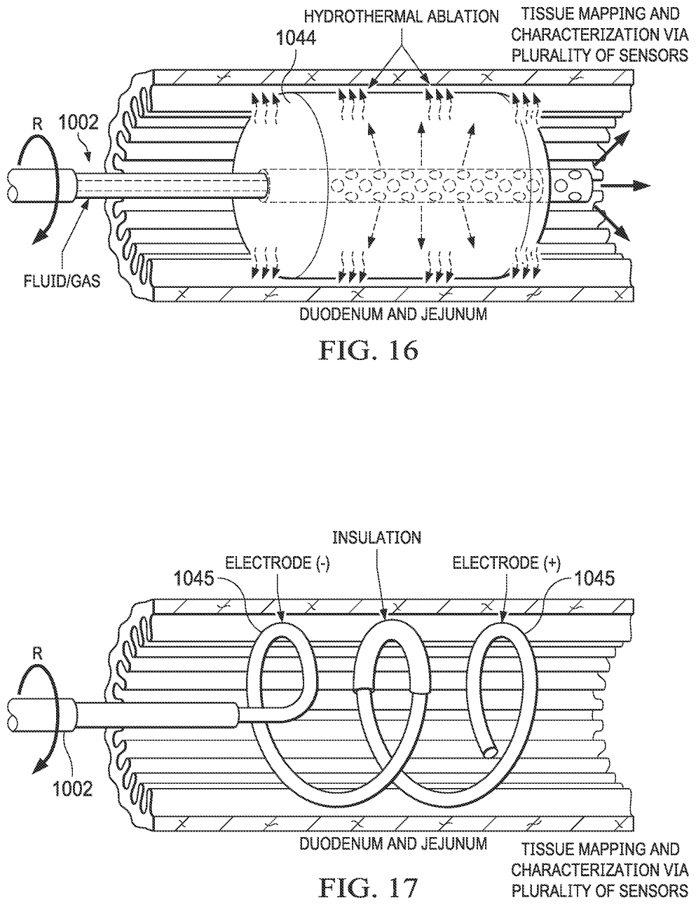

In some embodiments, hydrothermal ablation may be used to ablate the inner mucosal lining of the intestine with heated fluid filling a balloon 1044 delivered via the instrument working channel (see FIG. 16). The balloon can expand to make contact with the lumen wall, ablating the mucosal surface with heated temperatures. A feedback loop may be provided via a thermocouple or fiber optic temperature sensor. In some examples, axial rotation R of all or a portion of the device 1022 and/or the balloon 1044 within the anatomical lumen may be used for deployment and to optimize surface area contact. However, thermal application control by hydrothermal ablation may be limited. In some embodiments, a plurality of physical/mechanical, energy, chemical, or trans-mucosal injectate delivery devices (e.g., laser, needle, topical agent/chemical for trans-mucosal delivery and uptake, or microneedle application) extending directly from the balloon or adjacent to the balloon from a working channel may be used to inject a suspensory material (e.g. colloidal, hetastarch, saline, gas, combination thereof etc) in order to lift the mucosa layer from the underlying submucosal layer prior to ablation but after tissue mapping and characterization.

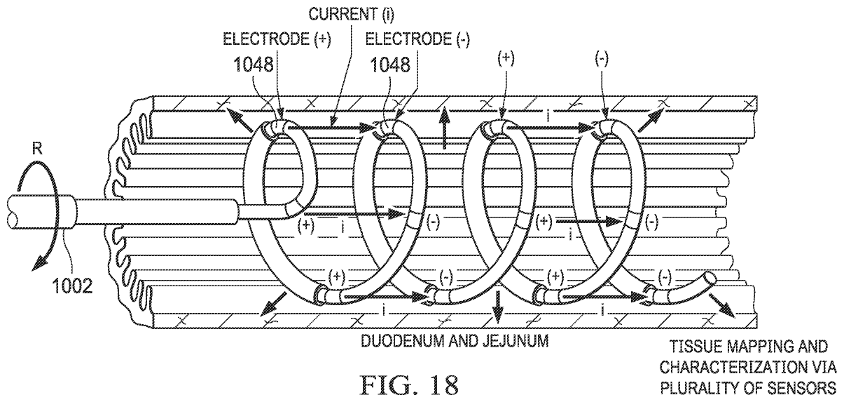

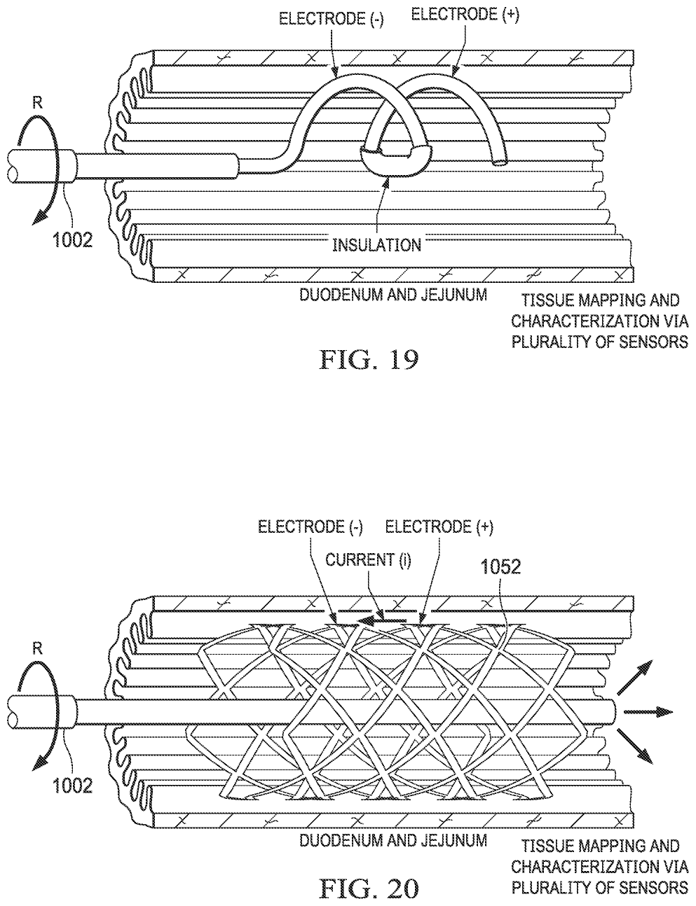

In some embodiments, radio frequency (RF) ablation is used to provide treatment providing better energy control than thermal ablation. The RF ablative energy may be applied via contact of the elongate device 1002 with the lumen wall surface at areas identified during the mapping step described above. Such ablative energy may be provided directly to the mucosa layer without applying a procedure to lift away from the submucosa as described above and below. Clinical feedback may be provided to measure effectiveness of the ablation treatment. e.g., using OCT for visualization to determine ablation depth. In some embodiments. RF energy using monopolar electrosurgery can be used but is prone to lateral spreading and to following blood vessels. Instead, improved control of thermal spread can be obtained by using bipolar electrosurgery providing RF energy selectively applied to a treatment area positioned between positive and negative electrodes. For example, an electrode or set of electrodes 1045 may be delivered to a treatment site by the elongate device 1002 in a collapsed configuration then expanded inside of the body lumen to contact the inner mucosal layer surface with electrodes deployed for direct energy application (see FIGS. 17-19). The electrodes may be mounted to an expandable member selectively deployable by the elongate device 1002 as the elongate device 1002 traverses the body lumens. In some embodiments, as shown in FIG. 18, multiple sets of electrode pairs (e.g., spaced-apart arc-shaped, semi-circular shaped, or ring-shaped pairs) may be selectively deployable by the elongate device 1002 inside of the body lumen to partially circumferentially apply energy to the inner mucosal layer surface. At least a portion of the electrode pairs may have an arcuate shape to contact and apply energy to a selected portion of the lumen circumference (see FIG. 19). Arc-shaped or semi-circular electrode pairs may then rotated and/or translated by the elongate device 1002 within the lumen to contact and apply energy to another selected portion of the lumen circumference to create a lesion spanning a larger circumference than a single ablation or several spaced non-circumferential lesions providing for ablation of a longer surface of the mucosal layer (i.e. higher treatment dose) while avoiding fully circumferential lesions which could result in lumen stenosis. In some embodiments when stenosis is not a concern, each of the electrode pairs may have the shape of ring (e.g., circle) as shown in FIG. 18, to contact and apply energy to the entire lumen circumference. In some embodiments, the electrodes may be located on the outside of an expandable member (e.g., a balloon or balloon catheter), and the electrodes may be located in a plurality of patterns on the outside of the expandable member (e.g., evenly distributed, helically distributed, radially, longitudinally, etc.).

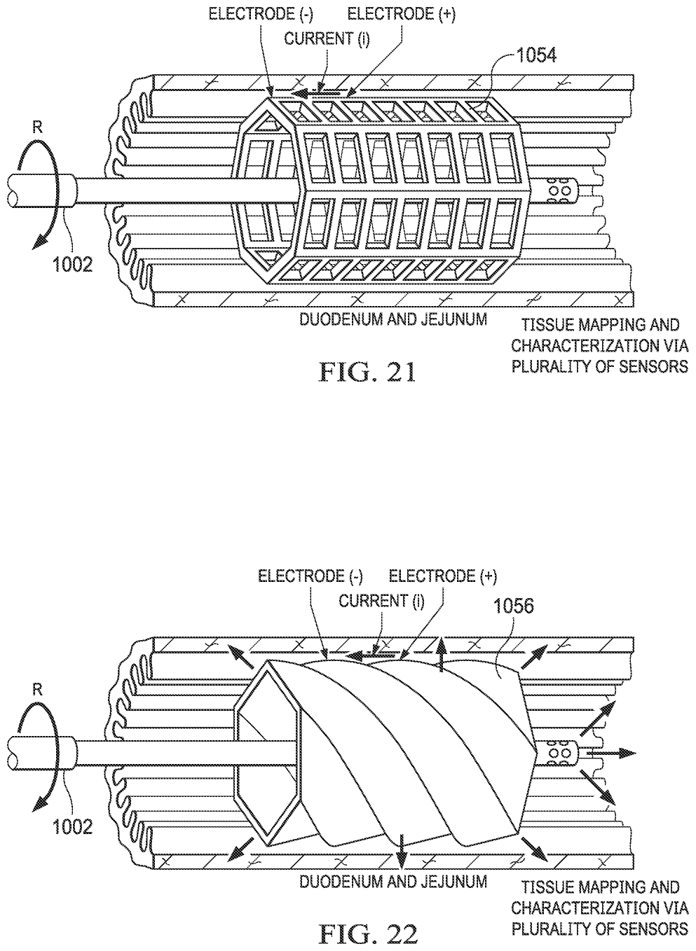

In some embodiments, as shown in FIGS. 20-22, an expandable/collapsible member (e.g., a mesh member, balloon, etc.) is provided that delivers ablative energy, wherein the expandable/collapsible member radially expands to contact the mucosal layer for ablation and radially contracts to move away from the mucosal layer to advance the expandable/collapsible member. The expandable/collapsible member allows for selective targeting of tissue in the radial direction and may be moved in a rotational direction to provide treatment around the circumference of the lumen. For example, a diamond-shaped, trapezoidal, rhomboidal, pentagonal, hexagonal, octagonal, or decagonal mesh member may be used. One or more sensors on the expandable/collapsible member (e.g., on a mesh member), positioned away from the direction of movement during treatment, may be applied to sense the completeness of the ablation. Further, a plurality of shapes (e.g., rhomboid, trapezoid, pentagon, hexagon, octagon, decagon, diamond, accordion-like, etc) may be used in order to increase surface area during deployment to more fully contact the mucosa. To protect the submucosal layer, the expandable/collapsible member optionally may have short microneedles that are used to inject fluid and/or gas into the submucosal layer to separate the mucosal and submucosal layers as described in more detail below. A control system optionally uses mucosal layer depth information as described above to gauge physical/mechanical, energy, chemical, or trans-mucosal injectate delivery (laser, needle, topical agent/chemical or microneedle insertion depth (e.g., pneumatic, hydraulic, or mechanical control of laser, needle, topical agent/chemical or microneedle length on the order of plus or minus one millimeter)).

Additional ablation mechanisms that may be used include plasma (both cold and hot) and sonic beam ultrasound (focal ultrasound therapy in combination with an external transducer). A plurality of plasma treatments may be used, including J plasma combining cold helium gas with radiofrequency energy, non-thermal (cold) plasma (partially ionized gas or gases comprising ions, electrons, ultraviolet photons and reactive neutrals such as radicals, excited, and ground-state molecules), argon plasma coagulation, and radiofrequency pulsed plasma (spray along the inside, then apply monopolar energy). The plasma may be delivered as a point or as a pinpoint, and automated delivery of plasma may be applied by advancing the elongate device 1002 while applying treatment for sub-millimeter depth targeted therapy.

In some embodiments, non-invasive, non-thermal, histosonic (sonic ultrasound beam ablation) of duodenal lining may be applied via a balloon deployed over a length of the duodenum with activation via an external transducer under real time visualization following mapping of enteroendocrine cell concentrations and surface area.

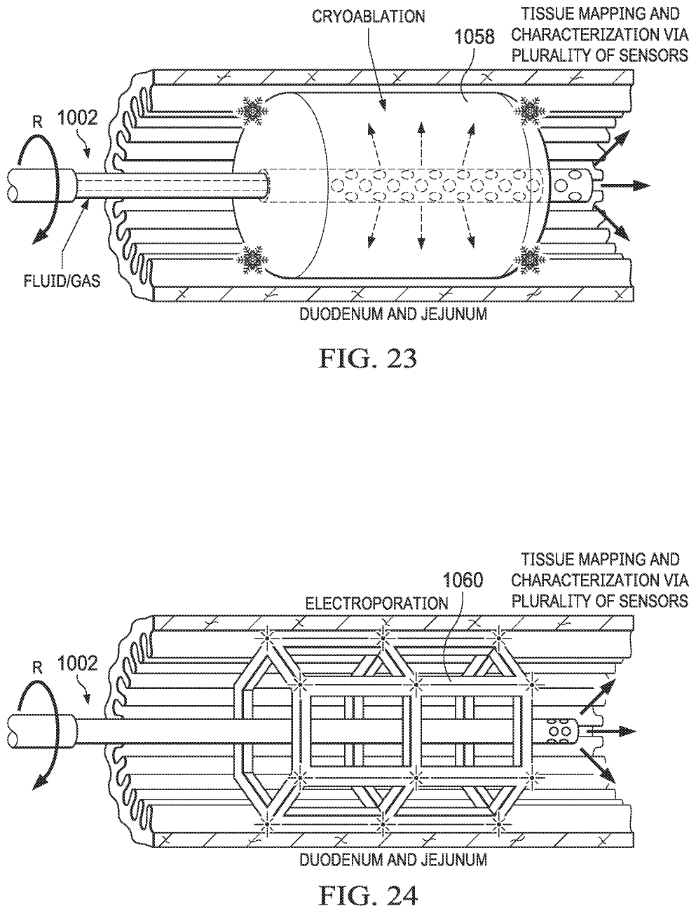

In some embodiments, use of cryoablation, cryosurgery, or cryotherapy may be used to ablate the inner mucosal lining of the intestine with cold gas filling a balloon delivered via the instrument working channel (see FIG. 23). The balloon can expand to make contact with the lumen wall, ablating the mucosal surface with freezing temperatures. A feedback loop may be provided via a thermocouple or fiber optic temperature sensor.

Treatment may be selectively provided to regions around the circumference of the duodenum lumen wall. For example, treatment may be applied only to the portion of the duodenum's inner circumference, or only along the outer curvature of the duodenum arch, and away from the sensitive areas. For example, in one embodiment approximately a 180-degree circumferential area of the duodenum sidewall along the outer portion of the C-curve section of the duodenum is treated, well away from identified sensitive structures. Other circumferential angular extents are possible in the C-curve section of the duodenum (e.g., 90 degrees, 120 degrees, 150 degrees, 210 degrees, 240 degrees, etc.) A larger or smaller circumferential area may optionally be treated in the region with sensitive structures. A larger circumferential area (e.g., up to and including 360 degrees) may optionally be treated immediately distal of the pyloric channel and/or distal of the duodenum's D1/D2 portion or C-curve to further increase the treatment area. In some embodiments, treatment may be selectively applied to a first portion of the circumference of a lumen wall (e.g., in the duodenum and/or jejunum) while a second portion of the circumference is not treated. Such selective circumferential treatment may be used to exclude a portion of the circumference from treatment, and may be used to protect sensitive areas from treatment. For example, treatment may be provided in circumferential regions of the duodenum around the ampulla of Vater and the papilla of Vater while avoiding treatment to these structures. For example, in some embodiments, an electrode (such as one or more of the electrodes in FIGS. 17-24) may allow for selective energization of portions of the circumference while preventing energization in areas of sensitivity. As another example, an expandable member that may be axially rotated for partial or full circumferential treatment (see FIGS. 17-19) may be controlled to restrict rotation in the areas of sensitivity. As another example, an expandable member having electrodes may have an electrode spacing that excludes portions of the circumference from treatment. For example, in some embodiments, crescent-shaped (e.g., arc-shaped) ablation is applied to selectively ablate one side of the duodenum wall lining. For example, a crescent-shaped instrument distal tip may perform treatment on one wall (e.g., the wall on the outer bend of the duodenum, using a 45-degree arc, 90-degree arc, 100-degree arc, or any suitable and desired alternative arc angle), and the tip may then be rotated towards another wall for treatment (e.g., the wall on the inner bend of the duodenum). In this way, the crescent-shaped ablation may be used to ablate all or a significant portion of the circumference of the duodenum wall proximal of the ampulla of Vater, only the portion of the circumference of the duodenum wall opposite the ampulla of Vater, and all or a significant portion of the circumference of the duodenum wall distal of the ampulla of Vater. As a result, a significant portion of the duodenum wall lining is ablated, and the sensitive areas of the duodenum wall are avoided. Device navigation and control with a computer-assisted teleoperated system as described herein is used for this aspect.

In some embodiments, electroporation may be used for treatment (see FIG. 24). Use of electroporation, or controlled electrical fields, may be used to target specific cells within the mucosal layer of the intestine, including enterocytes (aborptive cells), incretin hormone/endocrine producing cells (L/K enter-endocrine cells), cells of cajal contained within the stomach, goblet cells, and deeper paneth cells, etc.) by using a plurality of voltages, repetitions, pulses, and durations while avoiding damage to stem cells.

In some embodiments, photodynamic therapy may be used for treatment. For example, areas may be marked for treatment as described above (or areas may be selectively masked that are not desired for treatment), followed by using a light fiber to selectively remove the areas to be treated.

In some embodiments, use of a controlled electrical field may be used to prevent damage to intestinal muscle layer which contains fusiform-shaped myocytes. Using an electrical field, the contractile smooth muscle myocytes could be identified thus directing energy away from this layer of tissue via detection of the basal or basic electrical rhythm (BER) or electrical control activity (ECA) found within the stomach and intestines.

In some embodiments a helium-based argon coagulator may be used for treatment. Such treatment may involve a ring of electrodes in the lumen with gas being ejected out of nozzles. The gas is ignited to treat the areas adjacent the ignited gas.

In some embodiments, a sprayable hydrogel that causes mucosal necrosis may be used for treatment delivered via the instrument working channel.

In some embodiments, mechanical methods may be used for treatment. In some embodiments, the entire mucosa lining may be extracted followed by hemostasis with plurality of agents. In some embodiments, a set thickness of the mucosa may be cut or scraped with blades extending outward relative to the elongate device 1002. The blades may have a set cutting depth such that only a certain mucosa depth is cut. In some embodiments, a vacuum may be applied to lift the mucosa, and the top of the villa surface of the mucosa is then cut by using a mechanical blade. A blade may move in a circumferential direction, a longitudinal direction, or both directions in sequence or simultaneously. Irrigation and suction may be used to keep the area clear and prevent tissue build up. The volume in which mechanical ablation occurs may be insufflated to stretch the lumen wall to a reasonably flattened state to enhance the effectiveness of the mechanical ablation. Such insufflation may occur between two expandable balloons within the lumen so that mechanical ablation occurs on the inner surface of an insufflated body lumen cylinder between the two balloons that seal against gas leakage into proximal or distal portions of the body lumen. Coagulants may be used immediately following the mechanical ablation if needed, such as by spray application. In some embodiments, a hydro-jet utilizing water or fluid could be utilized to mechanically separate the inner mucosal lining via concentrated fluid pressure with or without heat to aid with coagulation and hemostasis.

With reference to FIG. 15B, in some embodiments, treatment may be enhanced via a submucosal lift procedure. In some embodiments, a partial or circumferential vacuum is applied to the mucosa to lift the mucosa layer, followed by ablation of the mucosal lining. In other embodiments, submucosal lift may be provided by using multiple circumferentially positioned needles in the elongate device 1002 in order to inject fluid 1043 (e.g., liquid and/or gasses) to dissect and isolate the inner, mucosal layer away from the submucosa. The liquid and/or gasses is injected into the submucosa region. Once the mucosal layer has been isolated away from the submucosa, larger depths of the mucosa region can be safely removed (e.g., via cutting or ablation as described above and below) while protecting the submucosa from removal. In some embodiments, sensors (e.g., resistance transducers) on the tips of selected or all needles may be used to gauge tissue depth and to provide feedback to the clinical user of being in the correct tissue plane for injection. Automated feedback and control may optionally adjust the needle depth based on the sensor output. Further, individual needle depths may be individually controlled for more effective dissection. In some embodiments, electrically insulating liquids and/or gasses may be injected into the submucosa layer to enhance energy-based ablation methods. The insulating liquids and/or gasses can serve as a further protective layer or region, which allows targeting treatment zones in the mucosa layer for removal via energy application (e.g., ablation energy) while the insulating layer hinders or prevents the energy from penetrating into the submucosa layer. The electrically insulating liquids and/or gasses may include purified water, de-ionized water, and/or lipids.

In some embodiments, treatment may be applied in multiple passes. In some embodiments, treatment may be overlapped (e.g., treatment over the same area more than once). In some embodiments, overlapping regions of treatment may be used with non-thermal energy applications. In some embodiments, non-overlapping treatment may be applied to prevent over-treatment. For example, to apply non-overlapping treatment, treatment may be applied, and then the elongate device 1002 is inserted or withdrawn a distance slightly more than the treatment area length such that a small untreated area remains between adjacent treated areas along the length of the body lumen.

In some embodiments, treatment is applied as the elongate device 1002 is advanced within the GI tract. In other embodiments, treatment is applied as the elongate device 1002 is retracted within the GI tract. In yet other embodiments, one or more portions of the treatment are applied as the elongate device 1002 is advanced, and one or more portions of the treatment are applied as the elongate device 1002 is retracted. For example, treatment may be applied by moving the elongate device 1002 to the desired distal limit of treatment and then retracting the elongate device 1002 proximally to administer treatment, which in contrast to insertion in some situations makes it easier to regulate a constant speed and/or distance during withdrawal.

In some embodiments, treatment is applied via rotation and/or translation of the elongate device 1002 in the lumen. For example, in some embodiments treatment is applied via simultaneous rotation and translation of the treatment portion of the device. In some embodiments the entire device rotates and/or translates. In some embodiments only the distal portion of the device that administers treatment or otherwise provides one or more sensing functions as described herein rotates and/or translates.

To further protect sensitive areas from treatment in some embodiments, areas of the stomach/duodenum/jejunum may be marked. For example, start and stop points may be marked in the duodenum designating areas to be treated by using options such as dyes/visual markers or implantable, leave-behind biodegradable sensors implanted during the surgery. The areas to be treated may be identified using sensors as described above. In some embodiments, sensing of peptide (e.g., GLP-1 and GIP peptide sensors; fluorescent, electrochemical, protein and peptide-based biosensors) and thus hormone readings may be provided to identify areas of mucosal hypertrophic overgrowth and/or entero-endocrine hyerplasia resulting in down-regulation/inhibition and thus decreased amounts of incretin hormones. A plurality of sensors (e.g., fluorescent, electrochemical, protein and peptide-based biosensors) to detect concentrations and thus mapping K and L cells within the mucosal lining or vasculature of the intestine may be provided. Such marking may be generated by a marking system and used by a control system (e.g., control system 812) to automatically prevent treatment to sensitive areas. For example, when using electrode-based treatment as described above, the control system may use the markers to selectively energize portions around the circumference while preventing energization in areas of sensitivity, and/or to restrict rotation of electrodes to prevent treatment in the areas of sensitivity. In greater detail, device localization data may be saved once an area of sensitivity has been identified. Then when re-encountering the location (e.g., recognized using localization data) after driving the device away and returning, a user may be notified (e.g., by audible and/or haptic warnings) when at or near the previously identified sensitive area. Alternatively, in an automated avoidance system, the control system not allow the user to move the device towards the marked sensitive areas and would stop motion of the device.

In some embodiments, laser speckle contrast imaging may be applied to image blood or other substance perfusion in treated regions to demarcate and visualize untreated regions. Laser speckle imaging provides a real time image to assess motion using red or near infrared light, and can provide sub surface information as to blood flow and thus the degree of tissue perfusion. The motion from peristalsis and camera movement may also be corrected for by increasing frame rates or by correcting for motion artifacts based on optical flow assessment from other sensors. This can be combined with a digital measurement tool to quantify dosage and treatment accuracy. In some examples, the digital measurement tool may measure the length of tissue ablated along the duodenum based on the margins detected from laser speckle contrast imaging. In some examples, these measurements may be visualized using overlays to warn the user regarding potential overlap of treated regions before a section is ablated.

Treatment may also be provided while avoiding the ampulla of Vater by using one or more methods. For example, visual indication to the clinical user may be provided in real time by using the imaging methods described above. For example, the ampulla of Vater may be manually or automatically marked or covered so as to not treat it. For example, a user may identify the ampulla of Vater by visual inspection using the imaging sensors. Optionally, the user may manually mark the ampulla of Vater on the structure itself and/or on a user display of the system. Optionally, use of AI/machine learning may be applied to identify and mark the ampulla of Vater. Further, identification of the ampulla with indocyanine green (ICG) administered intravenously (preoperatively or intraoperatively) may be applied, thus enabling identifying of the duodenal papilla in real time. In addition, enzymatic sensors may be used to detect bile acid or pancreatic enzyme presence from the ampulla of Vater (e.g., mass spectrometry probes as described above).

A pre-positioned flexible duodenal sleeve comprised of a biocompatible polymer may be used to prevent ablation to certain regions (e.g., around the ampulla of Vater). The sleeve is positioned against the inner wall of the duodenum such that ablation is not able to be performed where the sleeve is located. The sleeve may be solid, or the sleeve may be solid only on one side (e.g., webbed or meshed) for example to enable the lumen wall opposite the ampulla of Vater to be ablated while protecting the ampulla of Vater.

In some embodiments, an external sleeve around the duodenum and/or jejunum (e.g., inserted via a trans-abdominal port) may be provided to provide better electrical contact as treatment is provided. Conjunction of trans-abdominal positioning adjunct with computer-assisted teleperated instruments deployed via ports within the abdominal wall allows such instruments to be positioned around the outer lining of the duodenum and jejunum, thus aiding with positioning and measurements (e.g., combined endoscopic and computer-assisted laparoscopic duodenal therapy).

Tracking and/or Visualizing the Applied Treatment, the Area of Anatomy Treated, and Optionally Measuring and/or Characterizing Effectiveness

During a procedure and following treatment, it may be difficult to confirm that treatment has been applied to the target area and that desired non-treatment areas remain untreated. Further, it may be difficult to determine whether sufficient material has been removed during a procedure. It may be desirable to know the applied area of anatomy that has been treated and not treated, an amount of treatment being provided, and/or when to stop treatment during a procedure. It may be further desirable to know if and when a second treatment would be beneficial (e.g., at a later date). The present disclosure provides systems and multiple methods for providing such information.

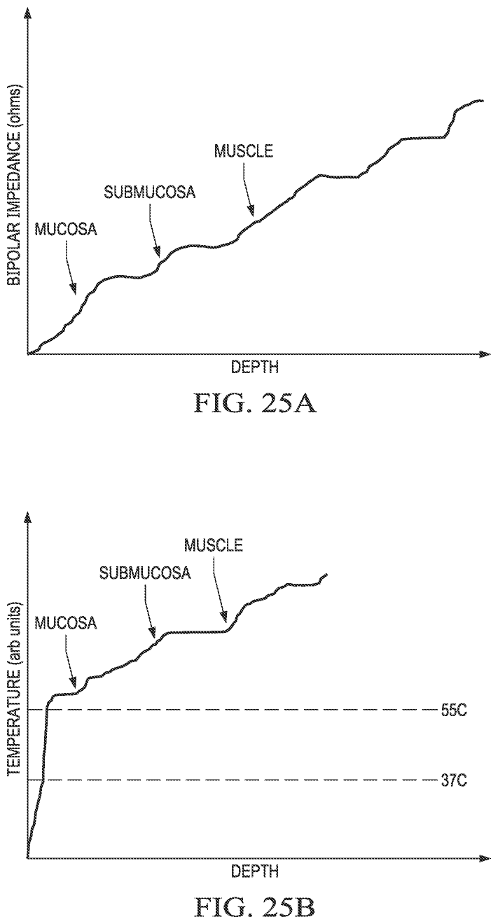

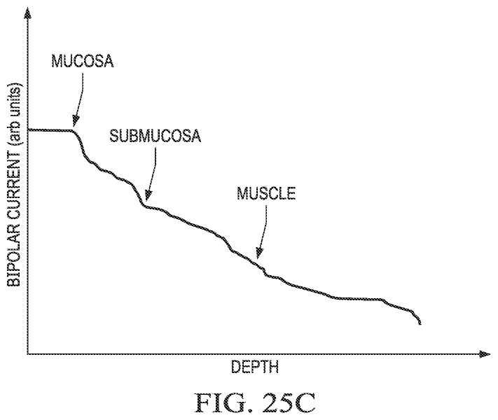

In some embodiments, various sensors may be used during the treatment procedure to track and/or visualize the area of anatomy treated and/or the applied treatment amount. Optionally, in some embodiments, the sensors may additionally or alternatively be used during the treatment to examine the effectiveness of the treatment. Imaging sensors (such as imaging sensors 1030 above) may be used to track and/or visualize indicators of ablation for the treated anatomy (e.g., tissue color changes, biochemical markers from cells that have been undergone treatment, or other products or byproducts of the ablation). In some embodiments, to track and visualize areas that have been treated, ablation may be applied by the methods described above, and the ablated region may be lightly marked or scored (e.g., by the same ablation treatment device) to indicate ablation has occurred. In some embodiments, tracking and/or visualizing may include marking the ablative locations on the tissue itself or marking a visual display based on the ablative locations that have been treated. The imaging sensors 1030 described above may be used to track and visualize the area of anatomy treated, the applied treatment amount, and/or optionally to characterize treatment effectiveness. Such sensors may also be referred to as efficacy sensors and may include mass spectrometry, spectroscopy, radar, nerve agents (fluorescent compound)/ICG, fluorescent endoscopy, hyperspectral/spectral imaging, OCT, radial ultrasound, temperature sensors, capsule positioning (leave behind) biodegradable sensors to identify areas treated (e.g., using stored kinematic distal tip positions as the tip moves to a target location), current sensors, impedance sensors, use of tissue impedance between the mucosal and submucosal layers to detect denaturation of tissue (coagulative necrosis of intestinal layers), and use of pH change after ablation to show that ablation has completed and to prevent double ablation. Sensor data from one or more of these sensors may be used to provide feedback to manually or automatically stop treatment. With reference to FIGS. 25A-25B, impedance and temperature rise as ablation depth increases, with different impedance and temperatures levels for the mucosal, submucosal, and muscle layers. Conversely, current decreases as ablation depth increases (see FIG. 25C). Sensor data from temperature, impedance, or current sensors may be used to predict the depth of applied ablation. Ablation may be manually or automatically stopped based on measuring a threshold level of impedance, temperature, and/or current. For temperature, the temperature of the musocal tissue should be heated beyond 50-55 C to cause cell death at the mucosal layer. The measured temperature may be used as a feedback control by the control system 122 to keep the tissue temperature at a pre-determined level to ensure proper ablation depth. In some embodiments using ablation for treatment, the proximity between the treatment device (e.g., electrode, balloon, etc.) and the tissue wall may be automatically varied based on impedance or temperature readings, readings from the navigation sensors 1006 and the imaging sensors 1030 described above, or from pre-operative mapping. Greater contact with the tissue wall may be applied for deeper ablation.

In some embodiments, use of HBE fluorescent markers with a capsule endoscope or capsule sensor is provided as a follow-up study to examine the extent of regrowth a certain time after the procedure (e.g., 6 weeks later).

One or more elements in embodiments of the present disclosure may be implemented in software to execute on a processor of a computer system such as control system 122/812. When implemented in software, the elements of the embodiments of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device, The code segments may be downloaded via computer networks such as the Internet. Intranet, etc.

Note that the processes and displays presented may not inherently be related to any particular computer or other apparatus. The required structure for a variety of these systems will appear as elements in the claims. In addition, the embodiments of the present disclosure are not described with reference to any particular programming language. It will be appreciated that a variety of programming languages may be used to implement the teachings of the disclosure as described herein.

While certain exemplary embodiments of the present disclosure have been described and shown in the accompanying drawings, it is to be understood that such embodiments are merely illustrative of and not restrictive on the broad inventive concept, and that the embodiments of the present disclosure not be limited to the specific constructions and arrangements shown and described, since various other modifications may occur to those ordinarily skilled in the art.

Various aspects of the subject matter described herein are set forth in the following numbered examples.

### Tissue Marking Examples

Example 1. A therapeutic method comprising:

characterizing tissue in a mucosal layer of a body lumen including characterizing the tissue as hypertrophic mucosal cells, hyperplastic mucosal cells, or sub-mucosal nerve cells;

identifying, based at least in part on the characterizing, target cells within the tissue;

ablating the target cells within the tissue; and

marking the tissue based on the ablation.

Example 2. The method of example 1 further comprising marking the tissue based on the identifying of the target cells.
Example 3. The method of example 1 or 2, wherein marking the tissue includes at least one of injecting dye, implanting biodegradable sensors, or further delivering ablation energy.
Example 4. The method of any of examples 1-8 further comprising:

determining an effectiveness of the ablation of the target cells.

Example 5. The method of any one of examples 1-4 further comprising:

creating a map of the body lumen; and

displaying the map including an indicator of the target cells within the wall of the body lumen.

Example 6. The method of example 5, wherein the indicator of the target cells represents identified target cells or ablated target cells
Example 7. The method of example 5 or 6, wherein:

the identifying of the target cells includes determining a first location of the target cells;

the first location is along a length of the body lumen; and

the indicator is displayed based on the first location.

Example 8. The method of examples 5 or 6, further comprising:

determining a second location of ablated target cells, wherein the second location is along a length of the body lumen and wherein the indicator is based on the second location.

Example 9. The method of any of examples 1-8, wherein the identifying includes determining a depth of the target cells within the mucosal layer.
Example 10. The method of example 9, wherein the determined depth is in a range of approximately 250-1500 micrometers.
Example 11. The method of any of examples 1-10, wherein the identifying of the target cells comprises detecting an increased amount of incretin hormones.
Example 12. The method of any of examples 1-11, further comprising:

identifying, based at least in part on the characterizing, sensitive anatomy along the body lumen, wherein the sensitive anatomy is at a third location along the body lumen.

Example 13. The method of any of examples 1-12 further comprising:

positioning a device within the body lumen at the target cells, wherein the device includes a sensor system for at least one of the characterizing of tissue, identifying of the target cells, ablating of the target cells, or the marking of the tissue and where the positioning of the device is based at least in part on the identification of the target cells.

### Device Anatomical Alignment Examples

Example 1. A medical method comprising:

determine, via at least one navigation sensor, proximity of a first portion of a medical device with an anatomical structure, wherein the medical device is positioned within a body lumen;

expanding, based on the detecting, an expandable member to contact a wall of the body lumen to align the first portion of the medical device with a longitudinal centerline of the body lumen;

rigidizing at least the first portion of the medical device;

collapsing the expandable member; and
receiving instructions to advance the medical device a total insertion distance.
   Example 2. The medical method of example 1, wherein the instructions for advancing the medical device include inserting a fixed distance, the fixed distance being an incremental distance of the total insertion distance.
   Example 3. The medical method of example 2 further comprising:

relaxing the first portion of the medical device after the medical device is inserted the fixed distance;

after relaxing the first portion, repeating the determining of the proximity of the first portion of the medical device with the anatomic structure, the expanding of the expandable element, the rigidizing of the first portion of the medical device, the collapsing of the expandable member, and the advancing of the medical device the incremental distance until the medical device has advanced the total insertion distance.

Example 4. The medical method of any of examples 1-3, wherein the determining of the proximity of the first portion of the medical device with the anatomic structure includes detecting a misalignment with the anatomic structure.
Example 5. The medical method of any of examples 1-4, wherein the anatomic structure is the wall of the body lumen.
Example 6. The medical method of example 5 further comprising: detecting a fluid meniscus to determine the proximity of the first portion of the medical device with the wall of the body lumen.
Example 7. The medical method of any of examples 1-4, wherein the anatomic structure is a body sphincter.
Example 8. The medical method of example 7, wherein the instructions for advancing the medical device a total insertion distance includes insertion of the medical device through the body sphincter.
Example 9. The medical method of example 7 or 8, wherein the determining of the proximity of the first portion of the medical device with the anatomic structure includes further detecting a pH gradient or pressure gradient in a direction of the body sphincter.
Example 10. The method of example 9, wherein the instructions for advancing the distal end of the medical device include moving the distal end towards the direction of the pH gradient or the direction of the pressure gradient.
Example 11. The method of any of claims 4-10 wherein detecting a misalignment with the anatomic structure comprises:

receiving data from at least one gravity sensor; and

calculating pose information of the medical device relative to the anatomic structure.

Example 12. The medical method of any of examples 1-11, wherein determining the proximity of the first portion of the medical device comprises:

detecting an orientation of the first portion of the medical device; and

determining the advancing of the medical device is deviates from the longitudinal centerline of the body lumen by a threshold amount.

Example 13. The medical method of any of examples 4-12 wherein the expanding of the expandable member is automatically triggered based on the determining detecting the misalignment of the medical device with the anatomic structure.
Example 14. The medical method of any of claims 4-13 further comprising providing user guidance based on the detecting of the misalignment of the medical device with the anatomic structure, wherein the user guidance is at least one of visual, audible, or haptic guidance.
Example 15. A medical method comprising:

detecting a fluid meniscus to determine a proximity of a first portion of a medical device with a wall of the body lumen; and

aligning the first portion of the medical device with a longitudinal centerline of the body lumen.

Example 16. A medical method comprising:

determining an alignment between a distal end of an instrument and a body sphincter, wherein the distal end of the instrument is positioned within a body lumen in proximity to the body sphincter;

providing instructions for aligning the distal end of the instrument with the body sphincter; and

providing instructions for inserting the distal end of the instrument through the body sphincter.

Example 17. The method of example 16, wherein:

determining the alignment includes detecting a pH gradient or a pressure gradient across the body sphincter; and

the instructions for inserting the distal end of the instrument include moving the distal end towards a direction of the pH gradient or a direction of the pressure gradient.

Example 18. The method of example 16 or 17, wherein the determining of alignment comprises:

receiving data from at least one gravity sensor; and

calculating pose information of the medical device relative to the body sphincter.

The method of Examples 1-18 may be performed by a system comprising a processor and a memory having computer readable instructions stored thereon. The computer readable instructions, when executed by the processor, cause the system to perform the method.

### Peristalsis Examples

Example 1. A system for delivering ablative energy comprising:

a medical device including a distal end portion;

an ablation device;

at least one anatomic motion sensor; and

a control system operatively coupled to the medical device, the ablation device, and the at least one anatomic motion sensor,

the control system comprising a processing system and a memory system operatively coupled to the processing system, wherein the memory system comprises programmed instructions adapted to cause the processing system to perform operations comprising:

detecting anatomic motion within the body lumen, wherein the motion occurs in at least one wave;

determining at least one window corresponding to the at least one wave; and

applying the ablative energy using the ablation device to the wall of the body lumen during the at least one window.

Example 2. The system of example 1, wherein the ablation device is coupled to the distal end portion of the medical device.
Example 3. The system of example 1 or 2, wherein the ablation device includes at least one of an RF electrode, microwave transducer, an ultrasound transducer, a heat element, a laser, a plasma energy delivery device, an electroporation electrode, a photosensitizer for delivering photodynamic therapy, a coagulator, or a hydrogel delivery mechanism.
Example 4. The system of any of examples 1-3, wherein the expandable element is a balloon.
Example 5. The system of any of examples 1-4, wherein the operations further comprise:

expanding an expandable device based on detecting the motion within the body lumen.

Example 6. The system of any of examples 1-5, wherein the anatomic motion sensor is coupled to the distal end portion of the medical device.
Example 7. The system of any of examples 1-6, wherein the anatomic motion sensor comprises one or more of a stretch sensor, a strain gauge transducer, a manometer, an accelerometer, an enteric nerve monitor, or an electromyographic monitor.
Example 8. The system of any of examples 1-7 further comprising an electric stimulation device for applying electric stimulation to the wall of the body lumen to initiate the anatomic motion within the body lumen.
Example 9. The system of example 8, wherein the electric stimulation device is coupled to the distal end portion of the medical device.
Example 10. The system of example 8 or 9, wherein the electric stimulation device is the ablation device.
Example 11. The system of any of examples 1-10, further comprising:

a user interface including an indicator representing the detected anatomic motion within the body lumen.

Example 12. The system of example 11, wherein the user interface comprises a display for providing the indicator.
Example 13. The system of example 11 or 12, wherein the user interface comprises an input device, wherein the indicator is haptic feedback provided through the input device.
Example 14. The system of any of examples 11-13, wherein the indicator is an audible indicator.
Example 15. The system of example 11, wherein the motion occurs in a first wave and a second wave, and wherein the window occurs between the first wave and the second wave.
Example 16. The system of example 11, wherein the window occurs during the at least one wave.
Example 17. The system of example 11, wherein the operations further comprise:

suspending the motion in the at least one wave, wherein the at least one window occurs during the suspension.

Example 18. The system of any of examples 1-17, wherein the at least one window occurs following the at least one wave and wherein the window includes a delay before the applying of the ablative energy.
Example 19. A system of any of examples 18, wherein the anatomic motion within the body lumen is caused by peristalsis and the at least one wave is a peristaltic wave.

### Selective Ablation Examples

Example 1. A method for selective ablation, the method comprising:

identifying target cells within a tissue wall for ablation based on data from at least one tissue characterization sensor; and

selectively ablating the target cells using a treatment device.

Example 2. The method of example 1, wherein the target cells for ablation are located along a first portion of a circumference of a body lumen and sensitive cells are located along a second portion of the circumference of the body lumen.
Example 3. The method of example 2, further comprising:

determining a first location of the target cells; and

determining a second location of the sensitive cells.

Example 4. The method of example 2 or 3, wherein selectively ablating the target cells includes protecting the sensitive cells.
Example 5. The method of any of examples 2-4, wherein the selectively ablating comprises:

expanding an expandable member to provide contact between the expandable member and the tissue wall, wherein the expandable member is coupled to the treatment device and wherein the expandable member includes at least one electrode coupled to the expandable member,

providing ablative energy using the at least one electrode to selectively ablate the target cells; and rotating the treatment device about a longitudinal axis of the treatment device, wherein the rotating includes restricting rotation to prevent ablation of sensitive cells.

Example 6. The method of any of examples 1-5, further comprising:

receiving data from at least one ablation feedback sensor, wherein the ablation feedback sensor includes at least a temperature sensor, an impedance sensor, or a current sensor.

Example 7. The method of example 6, further comprising determining a depth of ablation in the tissue wall based on at least one of a rise in impedance, a rise in temperature, or a decrease in current.
Example 8. The method of any of examples 1-6, wherein the target cells comprise at least one of hypertrophic mucosal cells, hyperplastic mucosal cells, or sub-mucosal nerve cells
Example 9. The method of any of examples 1-6, wherein the target cells are within a distal region of a duodenum or a proximal region of a jejunum within a patient intestine.
Example 10. The method of any of examples 1-9, wherein the at least one tissue characterization sensor comprises at least one of GLP-1 peptide sensors, GIP peptide sensors, narrow band imaging sensors, molecular level imaging sensors, confocal imaging sensors, hyperspectral imaging sensors, spectral imaging sensors, or photoacoustic imaging sensors.
Example 11. The method of any of examples 1-9, further comprising deploying a biodegradable capsule configured to contact an inner mucosal layer of the body lumen, the capsule comprising one or more of GLP-1 peptide sensors and GIP peptide sensors.
Example 12. The method of example 11, further comprising extending at least one needle from the biodegradable capsule into a submucosal layer surrounding an inner mucosal layer.

### Selective Ablation with Control System Examples

Example 1. A medical system for selective ablation, the system comprising:

a medical instrument including a treatment device and at least one tissue characterization sensor; and

a control system operably coupled to the medical instrument, wherein the control system comprises a processing system and a memory system operably coupled to the processing system, the memory system including programmed instructions adapted to cause the processing system to perform operations comprising:

identifying target cells within a tissue wall for ablation based on data from the at least one tissue characterization sensor; and

selectively ablating the target cells using the treatment device.

Example 2. The medical system of example 1, wherein the target cells for ablation are located along a first portion of a circumference of a body lumen and sensitive cells are located along a second portion of the circumference of the body lumen.
Example 3. The medical system of example 1, wherein the operations further comprise:

determining a first location of the target cells; and

determining a second location of the sensitive cells.

Example 4. The medical system of example 3, wherein selectively ablating the target cells includes protecting the sensitive cells.
Example 5. The medical system of any of example 1-4, wherein the treatment device comprises an expandable member and at least one electrode coupled to the expandable member, wherein the expandable member is configured for rotation about a longitudinal axis of the medical instrument.
Example 6. The medical system of example 5, wherein the expandable member is a hexagonal mesh or an octagonal mesh.
Example 7. The medical system of example 5, wherein:

the expandable member is a coil having an arcuate, semi-circular, or ring shape; and

the at least one electrode includes RF electrodes spaced along a length of the coil.

Example 8. The medical system of any of example 1-4, wherein the treatment device comprises a crescent-shaped distal tip, the tip being rotatable to contact the first portion of the circumference of the body lumen.
Example 9. The medical system of any of example 1-4, further comprising at least one ablation feedback sensor, wherein the ablation feedback sensor includes at least a temperature sensor, an impedance sensor, or a current sensor.
Example 10. The medical system of example 9, wherein the operations further comprise determining a depth of ablation in the tissue wall based on at least one of a rise in impedance, a rise in temperature, or a decrease in current.
Example 11. The medical system of any of example 1-4, wherein the target cells comprise at least one of hypertrophic mucosal cells, hyperplastic mucosal cells, or sub-mucosal nerve cells
Example 12. The medical system of any of example 1-4, wherein the at least one tissue characterization sensor include a peptide sensor or an imaging sensor.
Example 13. The medical system of example 12, wherein the at least one tissue characterization sensor comprise at least one of GLP-1 peptide sensors, GIP peptide sensors, narrow band imaging sensors, molecular level imaging sensors, confocal imaging sensors, hyperspectral imaging sensors, spectral imaging sensors, or photoacoustic imaging sensors.
Example 14. The medical system of example 12, wherein the at least one tissue characterization sensors includes a biodegradable capsule configured to contact an inner mucosal layer of the body lumen, the capsule comprising one or more of GLP-1 peptide sensors and GIP peptide sensors.
Example 15. The medical system of example 14, wherein the biodegradable capsule comprises needles configured to enter a submucosal layer surrounding the mucosal layer.

1. A medical system comprising:
at least one characterization sensor for providing data to identify an avoidance region of tissue along a body lumen wall;
an elongate device including a proximal end portion, a distal end portion and at least one electrical ablation element coupled to the distal end portion;
a marking system configured to mark the the avoidance region; and
a control system comprising a processor and a memory including machine readable instructions that, when executed by the processor, cause the control system to control activation of the electrical ablation element to prevent ablation in the marked avoidance region.

2. The medical system of claim 1, wherein the avoidance region includes a circumferential portion of the tissue along the body lumen wall.

3. The medical system of claim 1, wherein the marking system is further configured to mark the tissue in the body lumen wall identified by the at least one characterization sensor.

4. The medical system of claim 1, wherein the marking system includes a delivery mechanism for injecting dye or implantable sensors.

5. The medical system of claim 1, wherein the marking system includes the at least one ablation element, and wherein the at least one ablation element delivers a first amount of energy for ablating the tissue and a second amount of energy for marking the tissue.

6. The medical system of claim 1, wherein the at least one characterization sensor includes an efficacy sensor that provides data to determine effectiveness of the ablation.

7. The medical system of claim 6, wherein the efficacy sensor includes at least one selected from the group of an imaging sensor, a temperature sensor, a current sensor, an impedance sensor, a pH sensor, or a peptide sensor.

8. The medical system of claim 6, wherein the efficacy sensor is a camera for identifying a change in color of the tissue.

9. The medical system of claim 1 further comprising:
a display;
wherein the machine readable instructions, when executed by the processor, further cause the control system to
create a map of the body lumen wall;
determine a location of a plurality of target cells within the body lumen wall; and
display, on the display, the map including at least one indicator of the target cells at the location.

10. The medical system of claim 1, wherein the at least one characterization sensor configured to detect target cells in the tissue outside of the avoidance region.

11. The medical system of claim 10, wherein the at least one characterization sensor includes a depth sensor for determining a depth of the target cells within a plurality of layers of the tissue.

12. The medical system of claim 11 wherein the depth sensor includes an OCT sensor or an ultrasound sensor.

13. The medical system of claim 10, wherein the at least one characterization sensor for detecting the target cells includes a peptide sensor for detecting incretin hormones.

14. The medical system of claim 10, wherein the at least one characterization sensor for detecting the target cells includes an imaging sensor.

15. The medical system of claim 14, wherein the imaging sensor is a narrow band imaging sensor, molecular level imaging sensor, confocal imaging sensor, hyperspectral imaging sensor, spectral imaging sensor, or photoacoustic imaging sensor.

16. The medical system of claim 1, wherein the avoidance region includes sensitive anatomy along the body lumen wall.

17. The medical system of claim 1, wherein the body lumen wall surrounds a body lumen, the medical system further comprising:
an instrument manipulator for positioning the elongate device within the body lumen.

18-37. (canceled)

38. The medical system of claim 1, wherein the at least one electrical ablation element includes a plurality of electrodes configured to selectively energize portions of the tissue while preventing energization of the avoidance region.

39. The medical system of claim 38, wherein preventing energization of the avoidance region includes restricting a rotation of the plurality of electrodes.

40. The medical system of claim 1, wherein controlling control activation of the electrical ablation element includes providing a user notification when the distal end portion of the elongate device is at or near the avoidance region.
